(19) United States
(12) Patent Application Publication
(10) Pub. No.: US 2011/0165648 A1
(43) Pub. Date: Jul. 7, 2011

(54) Co-crystal structure of factor D and anti-factor D antibody

(76) Inventors: Menno Van Lookeren Campagne, San Francisco, CA (US); Christian Wiesmann, Bottmingen (CH)

(21) Appl. No.: 12/927,009

(22) Filed: Nov. 3, 2010

Related U.S. Application Data
(60) Provisional application No. 61/281,716, filed on Nov. 20, 2009.
(60) Provisional application No. 61/280,460, filed on Nov. 4, 2009.

(51) Int. Cl. C12N 9/96; C12N 9/64; G06G 7/58; G06F 17/14

(52) U.S. Cl. 435/188; 435/226; 703/11

(57) ABSTRACT

The present invention is directed towards the co-crystal structure of Factor D and an anti-Factor D antibody or an antigen binding fragment thereof.

### CROSS-REFERENCE TO RELATED APPLICATIONS

This application claims priority under 35 U.S.C. Section 119(e) and the benefit of U.S. Provisional Application Ser. Nos. 61/281,716 filed Nov. 20, 2009, and 61/280,460, filed Nov. 4, 2009, the contents of which are incorporated herein by reference in their entireties.

### FIELD OF THE INVENTION

The present invention concerns the co-crystal structure of Factor D and an anti-Factor D antibody or an antigen binding fragment thereof.

### BACKGROUND OF THE INVENTION

Factor D is a highly specific chymotrypsin-like serine protease that is a rate-limiting enzyme in the activation of the alternative complement pathway. The substrate for Factor D is another alternative pathway serine protease, factor B. Following cleavage by Factor D, factor B converts into the proteolytically active factor Bb and initiates the alternative complement pathway. Increased activation of the alternative complement pathway has been found in drusen. Drusen are cytotoxic complement-containing deposits present on the Bruch's membrane, which are associated with the development of age-related macular degeneration (AMD). A role of alternative pathway complement activation in AMD has further been supported by genetic analysis, showing that a mutation in factor H, a negative regulator of alternative complement pathway activation, is strongly correlated with increased risk for developing AMD.

Anti-Factor D antibodies are disclosed in U.S. Patent Publication Nos. 20080118506, published May 22, 2008; 20090181017, published Jul. 16, 2009; and 20090269338, published Oct. 29, 2009. Anti-Factor D antibodies find utility in the prevention and treatment of diseases and disorders associated with excessive or uncontrolled complement activation, and are useful for diagnostics, prophylaxis and treatment of disease.

### SUMMARY OF THE INVENTION

The instant disclosure presents the crystal structure of human and cynomolgous Factor D in complex with an anti-Factor D antibody fragment. The invention also provides information about the residues on human and cynomolgous Factor D that interact with the light- and heavy chains of the anti Factor D antibody Fab region.

In one aspect, the invention concerns a crystal formed by a native sequence Factor D polypeptide or a functional fragment or conservative amino acid substitution variant thereof.

In one embodiment, the native sequence Factor D polypeptide is human or cyno Factor D.

In another embodiment, the native sequence Factor D polypeptide is human Factor D of SEQ ID NO: 1.

In yet another embodiment, the crystal of human Factor D is characterized by unit cell

parameters approximately equal to the following: cell dimensions a=132.048; b=132.048; c=180.288; space group P4₃2₁2, crystal constant: 2.4 Å, and R/Rfree=21.2%/27.2.

In a further embodiment, the native sequence Factor D polypeptide is cyno Factor D of SEQ ID NO: 2.

In a still further embodiment, the crystal of cyno Factor D is characterized by unit cell parameters approximately equal to the following: a=182.205; b=80.673; c=142.575, space group C2, crystal constant: 2.1 Å; and R/Rfree=21.1%/26.9.

In another aspect, the invention concerns a Factor D crystal with the structural coordinates shown in Appendices 1A and 1B.

In yet, another aspect, the invention concerns a composition comprising any of the foregoing crystals.

In a different aspect, the invention concerns a crystallizable composition comprising a Factor D polypeptide complexed with an anti-Factor D antibody or an antigen binding fragment of said antibody.

In one embodiment, in the cystallizable composition the anti-Factor D antibody is a monoclonal antibody.

In another embodiment, the fragment is a Fab fragment.

In yet another embodiment, in the crystallizable composition the Factor D polypeptide is human Factor D of SEQ ID NO: 1.

In a further embodiment, in the crystallizable composition the Factor D polypeptide is cyno Factor D of SEQ ID NO: 2.

In a still further embodiment, the Factor D polypeptide comprises a catalytic triad.

In a further aspect, the invention concerns a crystal comprising a Factor D polypeptide complexed with an anti-Factor D antibody or an antigen binding fragment thereof.

In one embodiment, the antibody is a monoclonal antibody.

In another embodiment, the fragment is a Fab fragment.

In yet another embodiment, the Factor D polypeptide is human Factor D of SEQ ID NO: 1.

In a further embodiment, the Factor D polypeptide is cyno Factor D of SEQ ID NO: 2.

In a still further embodiment, the Factor D polypeptide comprises a catalytic triad.

In another embodiment, in the human Factor D polypeptide of SEQ ID NO: 1, or antigen binding fragment thereof, one or more of amino acid residues D131, V132, P134, D165, R166, A167, T168, N170, R171, R172, T173, D176, G177, I179, E181, R222, and K223 participate in complexing with the anti-Factor D antibody.

In yet another embodiment, in the human Factor D polypeptide of SEQ ID NO: 1, or antigen binding fragment thereof, all of amino acid residues D131, V132, P134, D165, R166, A167, T168, N170, R171, R172, T173, D176, G177, I179, E181, R222, and K223 participate in complexing with the anti-Factor D antibody.

In a different embodiment, in the human Factor D polypeptide of SEQ ID NO: 1, or antigen binding fragment thereof, amino acid residue R172 forms hydrogen bonds with the heavy and light chains of the anti-Factor D antibody of antigen binding fragment thereof.

In a different aspect, the invention concerns a computer for producing a three-dimensional representation of: a molecular complex comprising a binding site defined by structure coordinates of amino acid residues D131, V132, P134, D165, R166, A167, T168, N170, R171, R172, T173, D176, G177, I179, E181, R222, and K223 of human Factor D of SEQ ID NO: 1, wherein the computer comprises: (i) a machine-readable data storage medium comprising a data storage material encoded with machine-readable data, wherein said data comprises the structure coordinates of amino acid residues D131, V132, P134, D165, R166, A167, T168, N170, R171, R172, T173, D176, G177, I179, E181, R222, and K223 of human Factor D of SEQ ID NO: 1, and (ii) instructions for processing the machine-readable data into said three-dimensional representation.

In one embodiment, the computer further comprises a display for displaying said structure coordinates.

In another aspect, the invention concerns a method for evaluating the potential of a chemical entity to associate with a molecular complex comprising a binding site defined by structure coordinates of amino acid residues D131, V132, P134, D165, R166, A167, T168, N170, R171, R172, T173, D176, G177, I179, E181, R222, and K223 of human Factor D of SEQ ID NO: 1, comprising the steps of: (i) employing computational means to perform a fitting operation between the chemical entity and such binding site of the molecular complex; and (ii) analyzing the results of the fitting operation to quantify the association between the chemical entity and said binding site.

In one embodiment, the chemical entity is an antibody or an antigen binding fragment thereof, or a peptide mimetic or small molecule mimetic of the antibody or antibody fragment.

In another embodiment, the antibody, or antigen binding fragment thereof, forms hydrogen bonds with one or more of the listed residues.

In yet another embodiment, the antibody, or antigen binding fragment thereof, forms hydrogen bonds with amino acid residue R172 of human Factor D of SEQ ID NO: 1.

In a further aspect, the invention concerns chemical entities, such as antibodies, antibody fragments, peptide and small molecule mimetics identifiable or identified by the claimed methods.

In a still further aspect, the invention concerns a computer for determining at least a portion of the structure coordinates corresponding to an X-ray diffraction pattern of a molecular complex, wherein said computer comprises: a) a machine-readable data storage medium comprising a data storage material encoded with machine-readable data, wherein said data comprises at least a portion of the structure coordinates according FIGS. 6 and 7 or Appendix 1A or 1B; b) a machine-readable data storage medium comprising a data storage material encoded with machine-readable data, wherein said data comprises an X-ray diffraction pattern of said molecular complex; c) a working memory for storing instructions for processing said machine-readable data of a) and b); d) a central processing unit coupled to said working memory and to said machine-readable data of a) and b) for performing a Fourier transform of the machine readable data of (a) and for processing said machine readable data of (b) into structure coordinates; and e) a display coupled to said central processing unit for displaying said structure coordinates of said molecular complex.

### BRIEF DESCRIPTION OF THE DRAWINGS

FIG. 1. Crystal structure of human and cynomolgous Factor D (both in green) in complex with an anti Factor D Fab fragment (orange: heavy chain, yellow: light chain).

FIG. 2. Crystal structure of human and cynomolgous Factor D (in green) in complex with anti Factor D antibody Fab fragment (orange: heavy chain, yellow: light chain). Superposition is based on fD (the two respective molecules from each of the asymmetric units). This illustrates how similar the two cyno complexes are to each other and how similar the 2 human complexes are to each other.

FIG. 3. Superposition of all four Factor D:Fab complexes (two times cynomolgous (blue) and two times human (green). All structures are very similar with the exception of one area. A circle in the right figure marks were the cynomolgous and human Factor D structures slightly diverge.

FIG. 4. (a) Superposition of the 4 Factor D:Fab complexes (two times cynomolgous (blue) and two times human (green). The binding interface of all structures is identical. The histidine that forms part of the active site (circle in right figure) is in different conformations (human canonical, cyno inactive).

FIG. 5. Short-list of residues on Factor D (human) that interact with residues on the anti-Factor D antibody molecule.

FIGS. 6A and B. Residues on Factor D that interact with the light- and heavy chains of the anti Factor D Fab. Indicated in red are residues on the Fab heavy (H) and light (L) chain that form multiple hydrogen bonds with the key residue ARG-172 on human Factor D. 3 asterics (“***”) indicates that the OH forms a hydrogen bond.

The figures show the distance of every atom of Factor D (labeled as chain A) that is closer than 4.5 Å to any atoms of the Fab (labeled as chains L and H for light and heavy chains). For example:

| | | | | | |
|---|---|---|---|---|---|
| Asp 131A OD2 | . . . | TYR | 54H CE1 | . . . | 3.34 |
| | . . . | TYR | 54H CZ | . . . | 3.49 |
| | . . . | TYR | 54H OH | . . . | 2.78 *** |

means that the OD2 atom of Asp 131 in Factor D is close to 3 atoms of the Fab fragment. These three atoms are the

CE1 atom of Tyr 54 in the heavy chain (distance 3.34)

CZ atom of Tyr 54,

and the hydroxyl group of that tyrosine, with which Asp 131 of Factor D forms a hydrogen bond.

FIG. 7A. Key residue ARG-172 on human Factor D can potentially form 6 or more hydrogen ″1-bonds with heavy- and light-chain residues on the antibody.

FIG. 7B. Anti-Factor D Fab binds distant from the catalytic triad.

FIG. 8. Amino acid and nucleotide sequences of human Factor D (SEQ ID NOs: 1 and 3).

FIG. 9. Amino acid and nucleotide sequences of cyno Factor D (SEQ ID NOs: 2 and 4).

FIG. 10 depicts the amino acid sequences of the variable heavy chain and the variable light chain for each humanized antibody clone #56, #111, #250, and #416 (SEQ ID NOs: 5, 6, 7 and 8, respectively).

FIG. 11 shows the nucleotide sequence (SEQ ID NO: 9) of the light chain of humanized anti-Factor D Fab 238. The nucleotide sequence encodes for the light chain of humanized anti-Factor D Fab 238 with the start and stop codons shown in bold and underlined. The codon corresponding to the first amino acid in FIG. 11 (SEQ ID NO: 10) is bold and italicized.

FIG. 12 shows the amino acid sequence (SEQ ID NO: 10) of the light chain for humanized anti-Factor D Fab 238. The amino acid sequence lacks the N-terminus signal sequence of the polypeptide encoded by SEQ ID NO: 9 shown in FIG. 11. The HVR sequences are bold and italicized. Variable regions are regions not underlined while first constant domain CL1 is underlined. Framework (FR) regions and HVR regions are shown.

FIG. 13 shows the nucleotide sequence (SEQ ID NO: 18) of the heavy chain of humanized anti-Factor D Fab 238. The nucleotide sequence encodes for the heavy chain of humanized anti-Factor D Fab 238 with the start and stop codon shown in bold and underlined. The codon corresponding to the first amino acid in FIG. 14 (SEQ ID NO: 19) is bold and italicized.

FIG. 14 shows the amino acid sequence (SEQ ID NO: 19) of the heavy chain for humanized anti-Factor D Fab 238. The amino acid sequence lacks the N-terminus signal sequence of the polypeptide encoded by SEQ ID NO: 18 shown in FIG. 13. The HVR sequences are bold and italicized. Variable regions are regions not underlined while first constant domain CH1 is underlined. Framework (FR) regions and HVR regions are shown.

FIG. 15 shows the nucleotide sequence (SEQ ID NO: 28) of the light chain of humanized anti-Factor D Fab 238-1. The nucleotide sequence encodes for the light chain of humanized anti-Factor D Fab 238-1 with the start and stop codon shown in bold and underlined. The codon corresponding to the first amino acid in FIG. 16 (SEQ ID NO: 29) is bold and italicized.

FIG. 16 shows the amino acid sequence (SEQ ID NO 29) of the light chain for humanized anti-Factor D Fab 238-1. The amino acid sequence lacks the N-terminus signal sequence of the polypeptide encoded by SEQ ID NO: 28 shown in FIG. 15. The HVR sequences are bold and italicized. Variable regions are regions not underlined while first constant domain CL1 is underlined. Framework (FR) regions and HVR regions are shown.

FIG. 17 shows the nucleotide sequence (SEQ ID NO: 30) of the heavy chain of humanized anti-Factor D Fab 238-1. The nucleotide sequence encodes for the heavy chain of humanized anti-Factor D Fab 238-1 with the start and stop codon in bold and underlined. The codon corresponding to the first amino acid in FIG. 18 (SEQ ID NO: 31) is bold and italicized.

FIG. 18 shows the amino acid sequence (SEQ ID NO: 31) of the heavy chain for humanized anti-Factor D Fab 238-1. The amino acid sequence lacks the N-terminus signal sequence of the polypeptide encoded by SEQ ID NO: 30 shown in FIG. 18. The HVR sequences are bold and italicized. Variable regions are regions not underlined while first constant domain CH₁ is underlined. Framework (FR) regions and HVR regions are shown.

FIG. 19 shows the amino acid sequence of the light chain of anti-Factor D Fab 238-2 (SEQ ID NO: 40).

FIG. 20 shows the amino acid sequence of the heavy chain of anti-Factor D Fab 238-2 (SEQ ID NO: 41).

FIG. 21. Factor B cleavage is blocked by anti-Factor D antibody but not by 8E2 (fluid-phase assay)

FIGS. 22, 23. Factor D (S208A) binds to C3bB pro-convertase with an affinity of 772 nM (Biacore analysis).

FIGS. 24, 25 Anti-Factor D antibody blocks Factor D binding.

FIGS. 26, 27 Anti-Factor D antibody does not affect catalytic cleavage.

FIG. 28. Hypothetical model depicting how anti-Factor D antibody inhibits Factor B activation.

### DETAILED DESCRIPTION OF THE INVENTION

### I. Definitions

The terms “Factor D” and “complement Factor D” are used interchangeably, and refer to native sequence and variant Factor D polypeptides.

A “native sequence” Factor D, is a polypeptide having the same amino acid sequence as a Factor D polypeptide derived from nature, regardless of its mode of preparation. Thus, native sequence Factor D can be isolated from nature or can be produced by recombinant and/or synthetic means. In addition to a mature Factor D protein, such as the human Factor D protein of SEQ ID NO: 1, or the cyno Factor D protein of SEQ ID NO: 2, the term “native sequence Factor D”, specifically encompasses naturally-occurring precursor forms of Factor D (e.g., an inactive preprotein, which is proteolytically cleaved to produce the active form), naturally-occurring variant forms (e.g., alternatively spliced forms) and naturally-occurring allelic variants of Factor D, as well as structural conformational variants of Factor D molecules having the same amino acid sequence as a Factor D polypeptide derived from nature. Factor D polypeptides of non-human animals, including higher primates and non-human mammals, are specifically included within this definition, including but not limited to the cyno Factor D polypeptide of SEQ ID NO: 2.

“Factor D variant” or “complement Factor D variant” means an active Factor D polypeptide as defined below having at least about 80% amino acid sequence identity to a native sequence Factor D polypeptide, such as the native sequence human Factor D polypeptide of SEQ ID NO: 1, or the native sequence cyno Factor D polypeptide of SEQ ID NO: 2. Ordinarily, a Factor D variant will have at least about 80% amino acid sequence identity, or at least about 85% amino acid sequence identity, or at least about 90% amino acid sequence identity, or at least about 95% amino acid sequence identity, or at least about 98% amino acid sequence identity, or at least about 99% amino acid sequence identity with the mature human amino acid sequence of SEQ ID NO: 1 or the mature cyno Factor D polypeptide of SEQ ID NO: 2. Preferably, the highest degree of sequence identity occurs within the active site of Factor D.

The “active site” of Factor D is defined by His-57, Asp-102, and Ser-195 (chymotrypsinogen numbering) in the human Factor D sequence. Factor D has Asp189 (chymotrypsin numbering) at the bottom of the primary specificity pocket and cleaves an Arg peptide bond. The catalytic triad consists of His-57, Asp-102 and Ser-195. Asp-102 and His57 display atypical conformations compared with other serine proteases (Narayana et al., J. Mol. Biol. 235 (1994), 695-708). A unique sal bridge is observed between Asp189 and Arg218 at the bottom of the S1 pocket which elevated loop 214-218 and generated a deep and narrow S1 pocket (Jinget al., J. Mol. Biol. 282 (1998) 1061-1081). This loop and several other residues around the active site were shown by mutational analysis to be the key structural determinants of the Factor D esterolytic activity (Kim et al., J. Biol. Chem. 270 (1995) 24399-24405). Based on these results, it was proposed that Factor D may undergo a conformational change upon binding C3b-bound factor B, resulting in the expression of proteolytic activity (Volanakis and Narayana, Protein Sci. 5 (1996) 553-564).

As used herein, “solvent accessible position” refers to a position of an amino acid residue in the variable regions of the heavy and light chains of a source antibody or antigen binding fragment that is determined, based on structure, ensemble of structures and/or modeled structure of the antibody or antigen binding fragment, as potentially available for solvent access and/or contact with a molecule, such as an antibody-specific antigen. These positions are typically found in the CDRs and on the exterior of the protein. The solvent accessible positions of an antibody or antigen binding fragment, as defined herein, can be determined using any of a number of algorithms known in the art. Preferably, solvent accessible positions are determined using coordinates from a 3-dimensional model of an antibody, preferably using a computer program such as the InsightII program (Accelrys, San Diego, Calif.). Solvent accessible positions can also be determined using algorithms known in the art (e.g., Lee and Richards (1971) J. Mol. Biol. 55, 379 and Connolly (1983) J. Appl. Cryst. 16, 548). Determination of solvent accessible positions can be performed using software suitable for protein modeling and 3-dimensional structural information obtained from an antibody. Software that can be utilized for these purposes includes SYBYL Biopolymer Module software (Tripos Associates). Generally and preferably, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios (1994) Comput. Chem. 18(4): 377-386.

The term “binding pocket” refers to a region of a molecule or molecular complex, which, as a result of its shape, favorably associates with another chemical entity. The term “pocket” includes, but is not limited to, a cleft, channel or site. The shape of a binding pocket may be largely pre-formed before binding of a chemical entity, may be formed simultaneously with binding of a chemical entity thereto, or may be formed by the binding of another chemical entity thereto to a different binding pocket of the molecule, which in turn induces a change in shape of the binding pocket.

The term “generating a three-dimensional structure” or “generating a three-dimensional representation” refers to converting the lists of structure coordinates into structural models or graphical representation in three-dimensional space. This can be achieved through commercially or publicly available software. A model of a three-dimensional structure of a molecule or molecular complex can thus be constructed on a computer screen by a computer that is given the structure coordinates and that comprises the correct software. The three-dimensional structure may be displayed or used to perform computer modeling or fitting operations. In addition, the structure coordinates themselves, without the displayed model, may be used to perform computer-based modeling and fitting operations.

The term “crystallization solution” refers to a solution that promotes crystallization comprising at least one agent, including a buffer, one or more salts, a precipitating agent, one or more detergents, sugars or organic compounds, lanthanide ions, a poly-ionic compound and/or a stabilizer.

“Percent (%) amino acid sequence identity” is defined as the percentage of amino acid residues in a candidate sequence that are identical with the amino acid residues in a reference Factor D sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Sequence identity is then calculated relative to the longer sequence, i.e. even if a shorter sequence shows 100% sequence identity with a portion of a longer sequence, the overall sequence identity will be less than 100%.

“Percent (%) nucleic acid sequence identity” is defined as the percentage of nucleotides in a candidate sequence that are identical with the nucleotides in a reference Factor D-encoding sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Sequence identity is then calculated relative to the longer sequence, i.e. even if a shorter sequence shows 100% sequence identity with a portion of a longer sequence, the overall sequence identity will be less than 100%.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express an encoded polypeptide where, for example, the nucleic acid molecule is in a chromosomal location different from that of natural cells.

An “isolated” Factor D polypeptide-encoding nucleic acid molecule is a nucleic acid molecule that is identified and separated from at least one contaminant nucleic acid molecule with which it is ordinarily associated in the natural source of the Factor D-encoding nucleic acid. An isolated Factor D polypeptide-encoding nucleic acid molecule is other than in the form or setting in which it is found in nature. Isolated Factor D polypeptide-encoding nucleic acid molecules therefore are distinguished from the encoding nucleic acid molecule(s) as they exists in natural cells. However, an isolated Factor D-encoding nucleic acid molecule includes Factor D-encoding nucleic acid molecules contained in cells that ordinarily express Factor D where, for example, the nucleic acid molecule is in a chromosomal location different from that of natural cells.

The term “antagonist” is used in the broadest sense, and includes any molecule that is capable of neutralizing, blocking, partially or fully inhibiting, abrogating, reducing or interfering with a Factor D biological activity. Factor D antagonists include, without limitation, anti-Factor D antibodies and antigen binding fragments thereof, other binding polypeptides, peptides, and non-peptide small molecules, that bind to Factor D and are capable of neutralizing, blocking, partially or fully inhibiting, abrogating, reducing or interfering with Factor D activities, such as the ability of Factor D to participate in the pathology of a complement-associated eye condition.

A “small molecule” is defined herein to have a molecular weight below about 600, preferably below about 1000 daltons.

“Active” or “activity” or “biological activity” in the context of a Factor D antagonist of the present invention is the ability the antagonize (partially or fully inhibit) a biological activity of Factor D. A preferred biological activity of a Factor D antagonist is the ability to achieve a measurable improvement in the state, e.g. pathology, of a Factor D-associated disease or condition, such as, for example, a complement-associated eye condition. The activity can be determined in in vitro or in vivo tests, including binding assays, using a relevant animal model, or human clinical trials.

The term “complement-associated eye condition” is used in the broadest sense and includes all eye conditions the pathology of which involves complement, including the classical and the alternative pathways, and in particular the alternative pathway of complement. Complement-associated eye conditions include, without limitation, macular degenerative diseases, such as all stages of age-related macular degeneration (AMD), including dry and wet (non-exudative and exudative) forms, choroidal neovascularization (CNV), uveitis, diabetic and other ischemia-related retinopathies, and other intraocular neovascular diseases, such as diabetic macular edema, pathological myopia, von Hippel-Lindau disease, histoplasmosis of the eye, Central Retinal Vein Occlusion (CRVO), corneal neovascularization, and retinal neovascularization. A preferred group of complement-associated eye conditions includes age-related macular degeneration (AMD), including non-exudative (wet) and exudative (dry or atrophic) AMD, choroidal neovascularization (CNV), diabetic retinopathy (DR), and endophthalmitis.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In treatment of an immune related disease, a therapeutic agent may directly alter the magnitude of response of a component of the immune response, or render the disease more susceptible to treatment by other therapeutic agents, e.g., antibiotics, antifungals, anti-inflammatory agents, chemotherapeutics, etc.

The “pathology” of a disease, such as a complement-associated eye condition, includes all phenomena that compromise the well-being of the patient. This includes, without limitation, abnormal or uncontrollable cell growth (neutrophilic, eosinophilic, monocytic, lymphocytic cells), antibody production, auto-antibody production, complement production, interference with the normal functioning of neighboring cells, release of cytokines or other secretory products at abnormal levels, suppression or aggravation of any inflammatory or immunological response, infiltration of inflammatory cells (neutrophilic, eosinophilic, monocytic, lymphocytic) into cellular spaces, etc.

The term “mammal” as used herein refers to any animal classified as a mammal, including, without limitation, humans, higher primates, domestic and farm animals, and zoo, sports or pet animals such horses, pigs, cattle, dogs, cats and ferrets, etc. In a preferred embodiment of the invention, the mammal is a human.

Administration “in combination with” one or more further therapeutic agents includes simultaneous (concurrent) and consecutive administration in any order.

“Therapeutically effective amount” is the amount of a “Factor D antagonist” which is required to achieve a measurable improvement in the state, e.g. pathology, of the target disease or condition, such as, for example, a complement-associated eye condition.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42C; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a polypeptide of the invention fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

The term “antibody” is used in the broadest sense and specifically covers, without limitation, single anti-Factor D monoclonal antibodies (including agonist, antagonist, and neutralizing antibodies) and anti-Factor D antibody compositions with polyepitopic specificity. The term “monoclonal antibody” as used herein refers to an antibody obtained from a population of substantially homogeneous antibodies, i.e., the individual antibodies comprising the population are identical except for possible naturally-occurring mutations that may be present in minor amounts.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81:6851-6855).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596.

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (k_{d}) value of no more than about 1×10⁻⁷ M, preferably no more than about 1×10⁻⁸ M and most preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

As used herein, “antibody mutant” or “antibody variant” refers to an amino acid sequence variant of the species-dependent antibody wherein one or more of the amino acid residues of the species-dependent antibody have been modified. Such mutants necessarily have less than 100% sequence identity or similarity with the species-dependent antibody. In a preferred embodiment, the antibody mutant will have an amino acid sequence having at least 75% amino acid sequence identity or similarity with the amino acid sequence of either the heavy or light chain variable domain of the species-dependent antibody, more preferably at least 80%, more preferably at least 85%, more preferably at least 90%, and most preferably at least 95%. Identity or similarity with respect to this sequence is defined herein as the percentage of amino acid residues in the candidate sequence that are identical (i.e same residue) or similar (i.e. amino acid residue from the same group based on common side-chain properties, see below) with the species-dependent antibody residues, after aligning the sequences and introducing gaps, if necessary, to achieve the maximum percent sequence identity. None of N-terminal, C-terminal, or internal extensions, deletions, or insertions into the antibody sequence outside of the variable domain shall be construed as affecting sequence identity or similarity.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino kid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, “antibody variable domain” refers to the portions of the light and heavy chains of antibody molecules that include amino acid sequences of Complementarity Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, the term “Complementarity Determining Regions (CDRs; ie., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5^{th} Ed. Public Health Service, National Institutes of Health, Bethesda, Md. (1991)) and/or those residues from a “hypervariable loop” (i.e. about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk (1987) 1 Mol. Biol. 196:901-917). In some instances, a complementarity determining region can include amino acids from both a CDR region defined according to Kabat and a hypervariable loop. For example, the CDRH1 of the heavy chain of antibody 4D5 includes amino acids 26 to 35.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

As used herein, “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein. A codon set typically is represented by 3 capital letters in italics, eg. NNK, NNS, XYZ, DVK and the like. A “non-random codon set”, as used herein, thus refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al. (1999) J. Mol. Biol. 296:57-86); Garrard & Henner (1993) Gene 128:103). Such sets of oligonucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can, but does not necessarily, include restriction enzyme sites useful for, for example, cloning purposes.

The term “antibody fragment” is used herein in the broadest sense and includes, without limitation, Fab, Fab′, F(ab′)₂, scFv, (scFv)₂, dAb, and complementarity determining region (CDR) fragments, linear antibodies, single-chain antibody molecules, minibodies, diabodies, and multispecific antibodies formed from antibody fragments.

An “Fv” fragment is an antibody fragment which contains a complete antigen recognition and binding site. This region consists of a immer of one heavy and one light chain variable domain in tight association, which can be covalent in nature, for example in scFv. It is in this configuration that the three CDRs of each variable domain interact to define an antigen binding site on the surface of the V_{H}—V_{L} immer. Collectively, the six CDRs or a subset thereof confer antigen binding specificity to the antibody. However, even a single variable domain (or half of an Fv comprising, only three CDRs specific for an antigen) has the ability to recognize and bind antigen, although usually at a lower affinity than the entire binding site.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen binding sites. Diabodies are described more fully in, for example, EP 404,097; WO 93/11161; and Hollinger et al. (1993) Proc. Natl. Acad. Sci. USA 90:6444-6448.

The expression “linear antibodies” refers to the antibodies described in Zapata et al. (1995 Protein Eng, 8(10):1057-1062). Briefly, these antibodies comprise a pair of tandem Fd segments (V_{H}—C_{H}1-V_{H}—C_{H}1) which, together with complementary light chain polypeptides, form a pair of antigen binding regions. Linear antibodies can be bispecific or monospecific.

As used herein, “library” refers to a plurality of antibody or antibody fragment sequences (for example, polypeptides of the invention), or the nucleic acids that encode these sequences, the sequences being different in the combination of variant amino acids that are introduced into these sequences according to the methods of the invention.

“Phage display” is a technique by which variant polypeptides are displayed as fusion proteins to at least a portion of coat protein on the surface of phage, e.g., filamentous phage, particles. A utility of phage display lies in the fact that large libraries of randomized protein variants can be rapidly and efficiently sorted for those sequences that bind to a target antigen with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman (1992) Curr. Opin. Struct. Biol. 3:355-362, and references cited therein. In a monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells (1991) Methods: A companion to Methods in Enzymology 3:205-0216.

A “phagemid” is a plasmid vector having a bacterial origin of replication, e.g., Co1E1, and a copy of an intergenic region of a bacteriophage. The phagemid may be used on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle.

The term “phage vector” means a double stranded replicative form of a bacteriophage containing a heterologous gene and capable of replication. The phage vector has a phage origin of replication allowing phage replication and phage particle formation. The phage is preferably a filamentous bacteriophage, such as an M13, fl, fd, Pf3 phage or a derivative thereof, or a lambdoid phage, such as λ, 21, phi80, phi81, 82, 424, 434, etc., or a derivative thereof.

The terms “peptide mimetic” and “peptidomimetic” are used interchangeably, and refer to conformationally well defined peptide molecules, that mimic the structures and binding properties of a Factor D recognition region (epitope) of an anti-Factor D antibody herein. The crystal structures herein enable the identification and preparation of such peptide mimetics.

### DETAILED DESCRIPTION

Crystal Structure and Molecular Modeling

Applicants have solved the three-dimensional structure of a Factor D/anti-Factor D antibody complex using high resolution X-ray crystallography. This work has provided, for the first time, information about the binding site of Factor D for an anti-Factor D antibody, and of the residues of an anti-Factor D antibody heavy and light chains participating in binding to Factor D.

In one aspect, the invention concerns crystallizable compositions comprising a Factor D polypeptide complexed with an anti-Factor D antibody or an antigen binding fragment of such antibody.

The crystallizable compositions provided by the present invention are amenable to X-ray crystallography. Therefore, this invention also encompasses crystals of the crystallizable compositions.

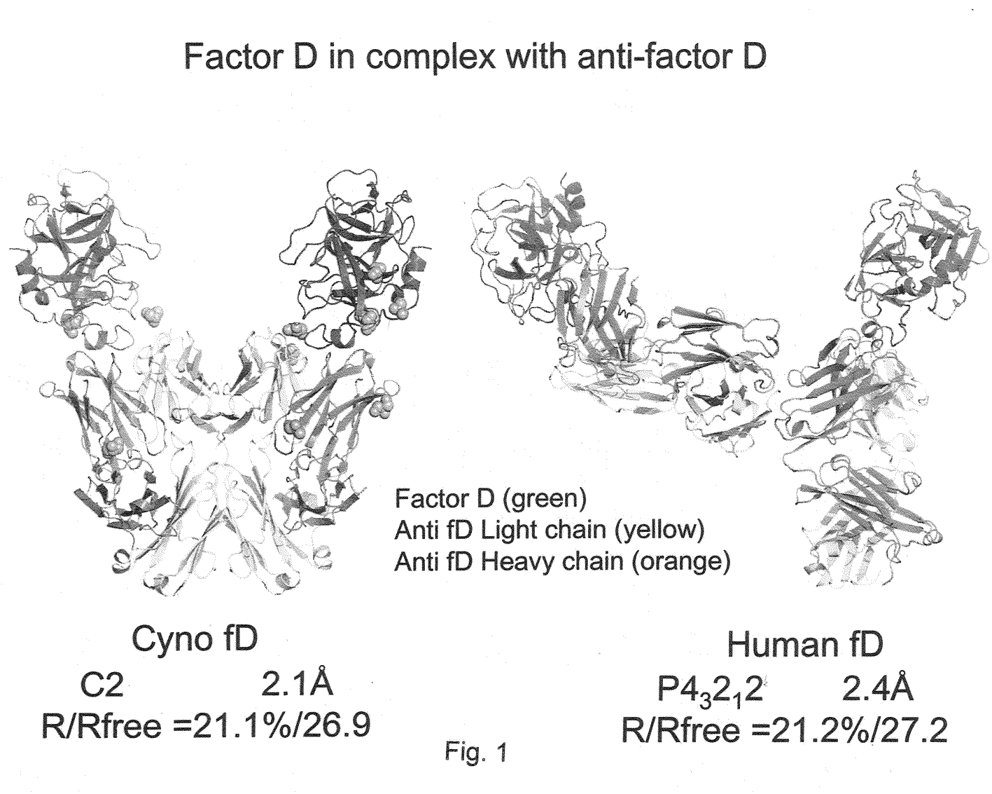

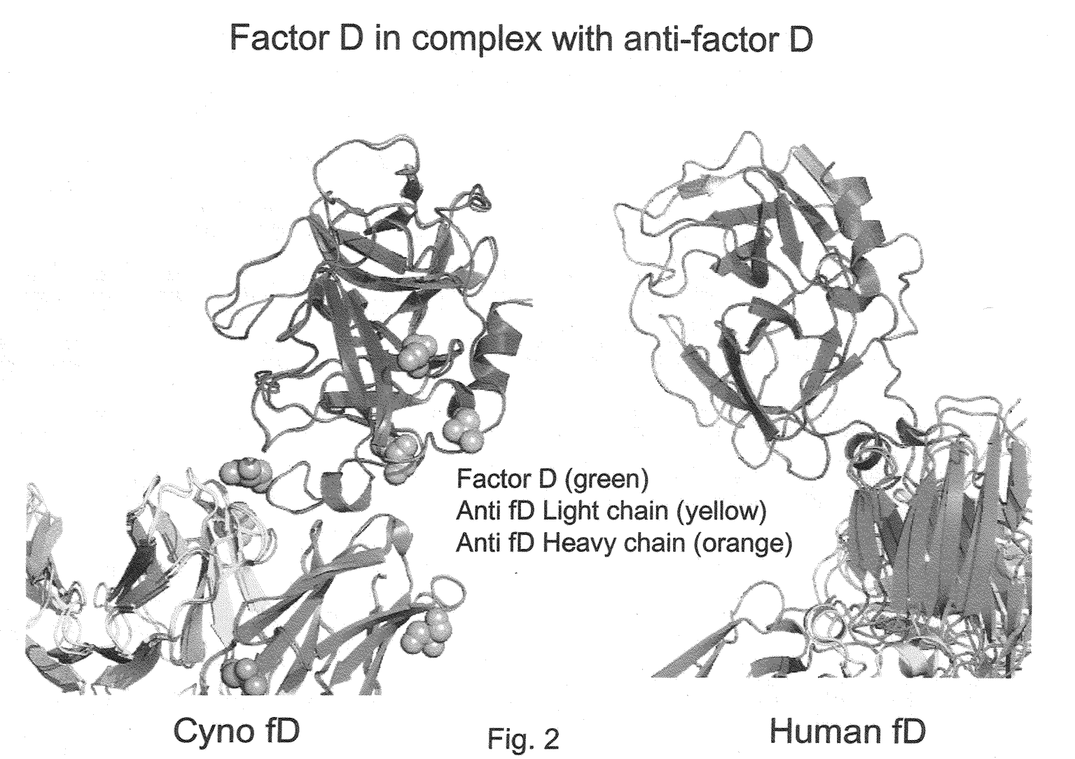

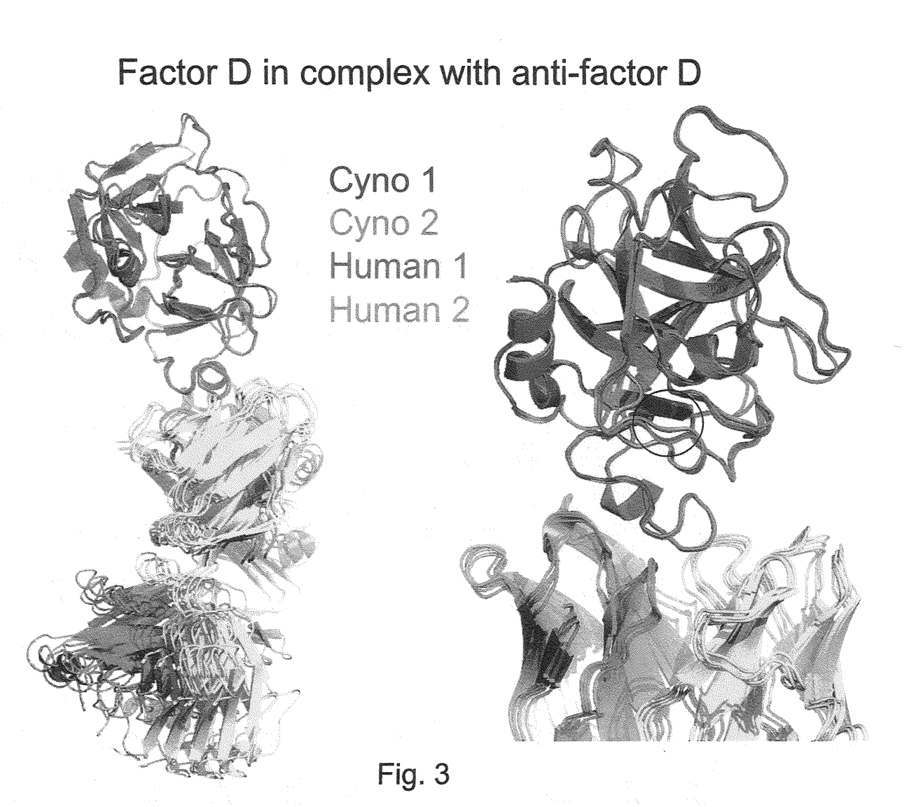

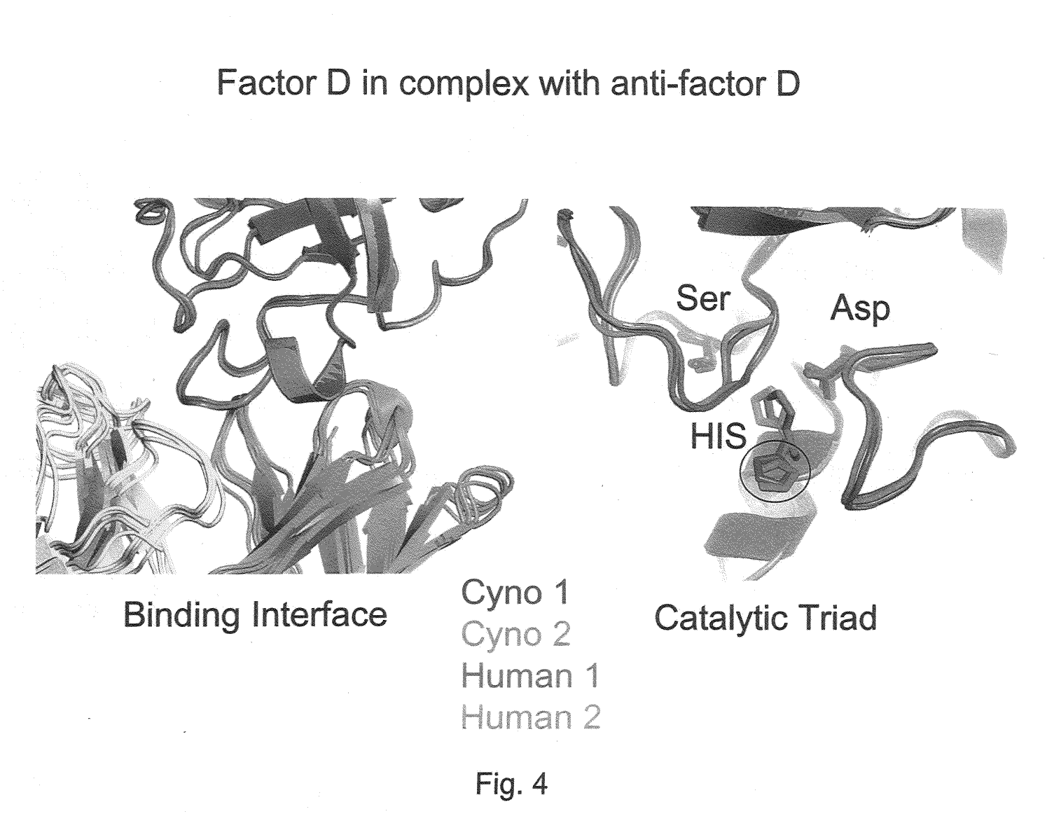

This invention further provides the three dimensional structure of a Factor D/anti-Factor D antibody complex at high resolution (e.g. 2.1 Å or 2.4 Å resolution, see FIG. 1)

X-ray crystallographic techniques are known in the art. The three-dimensional structure of the Factor D/anti-Factor D antibody complex is defined by a set of structure coordinates as set forth in Appendices 1A and 1B. The term “structure coordinates” refers to Cartesian atomic coordinates derived from mathematical equations related to the patterns obtained on diffraction of a monochromatic beam of X-rays by the atoms of an extracellular domain of a Factor D/anti-Factor D antibody complex in crystalline form.

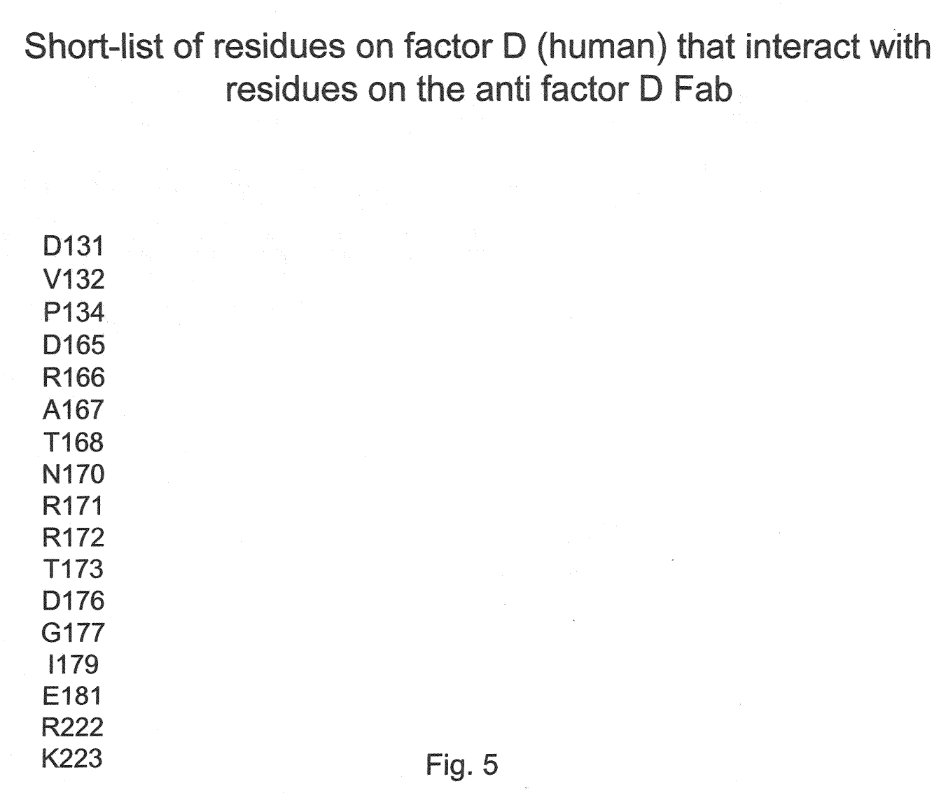

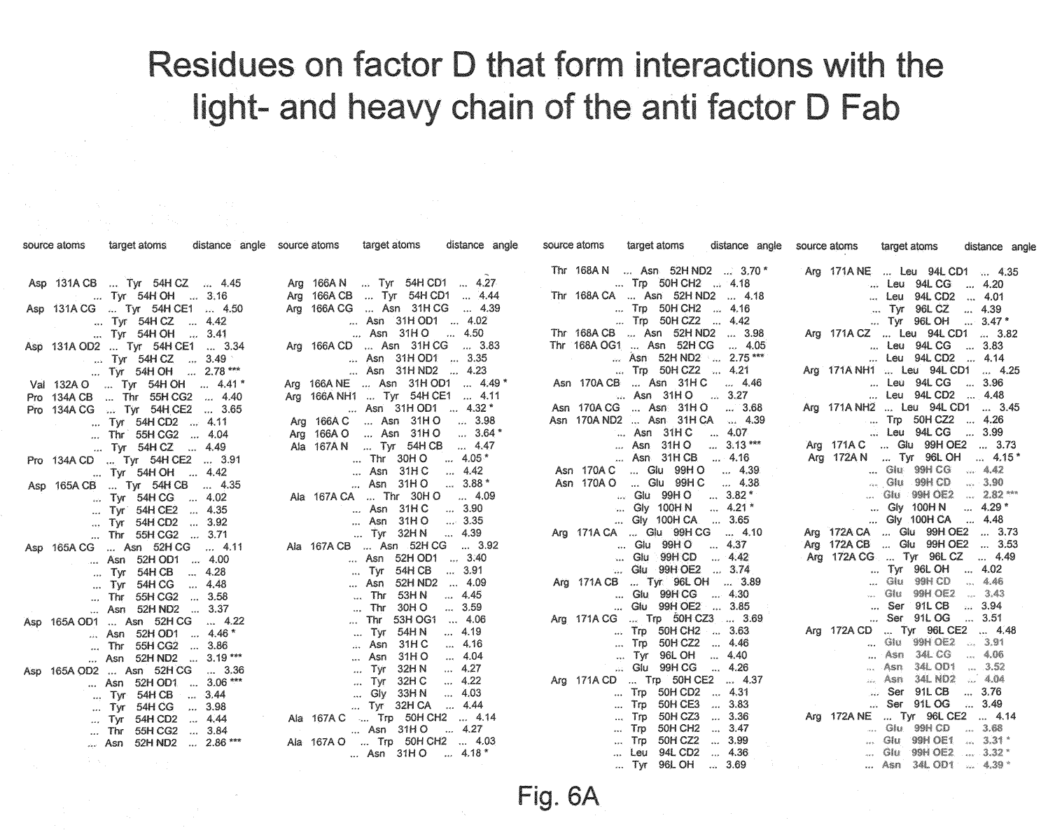

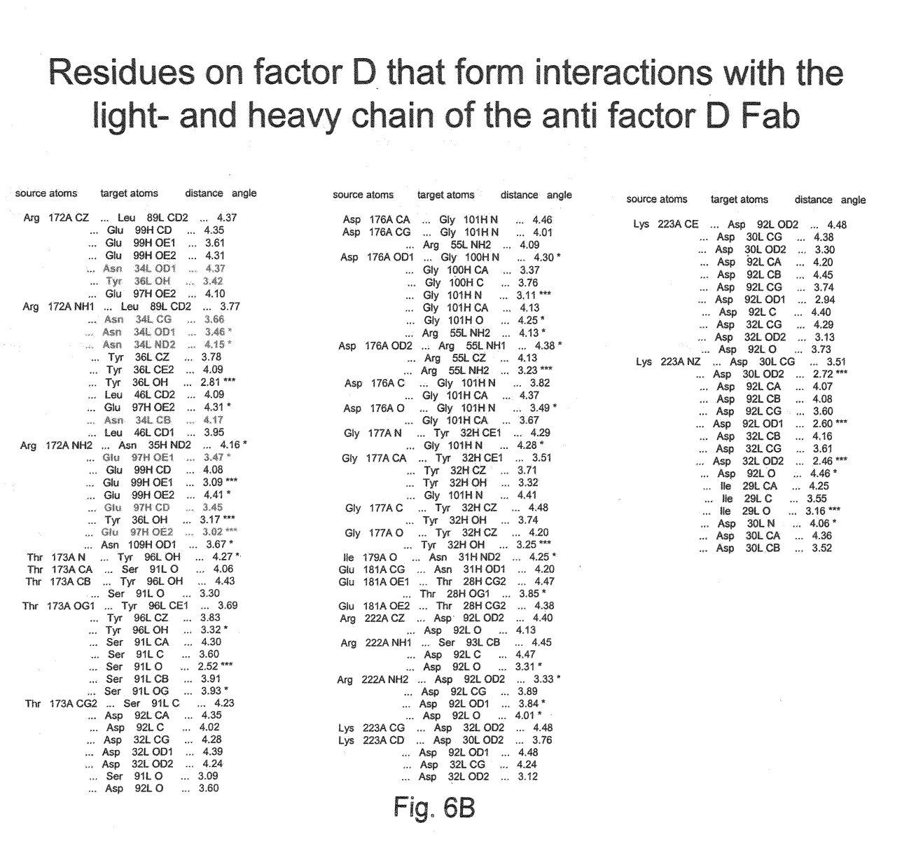

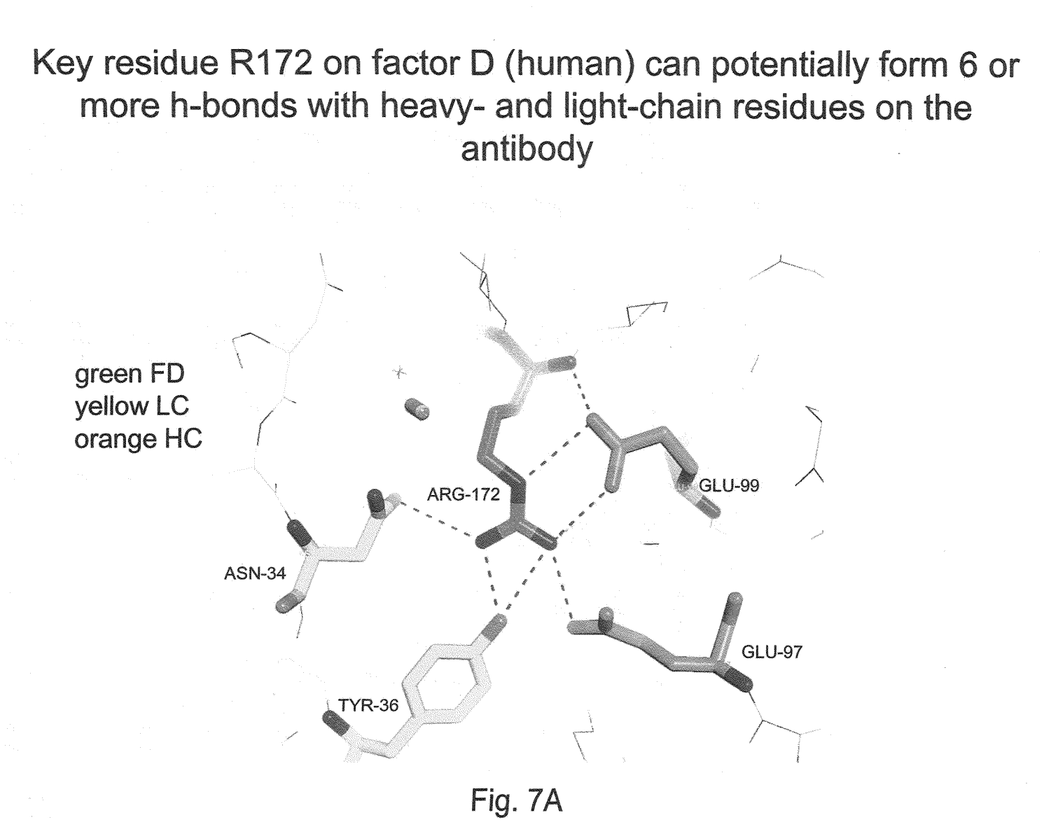

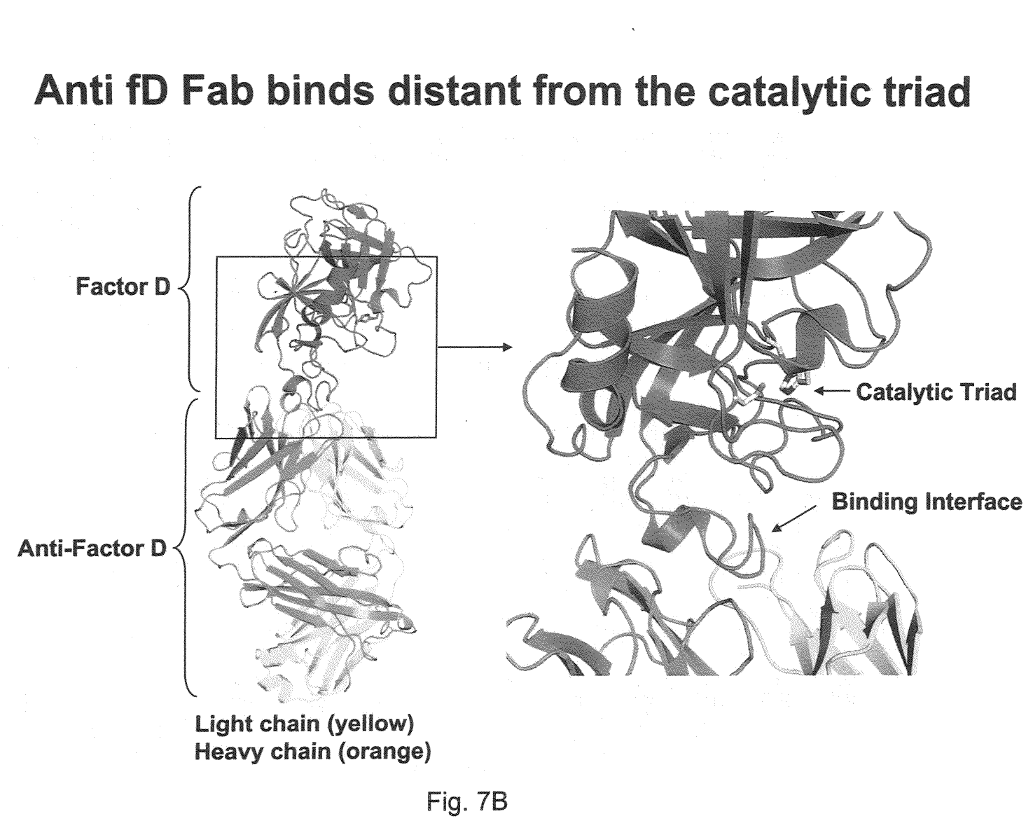

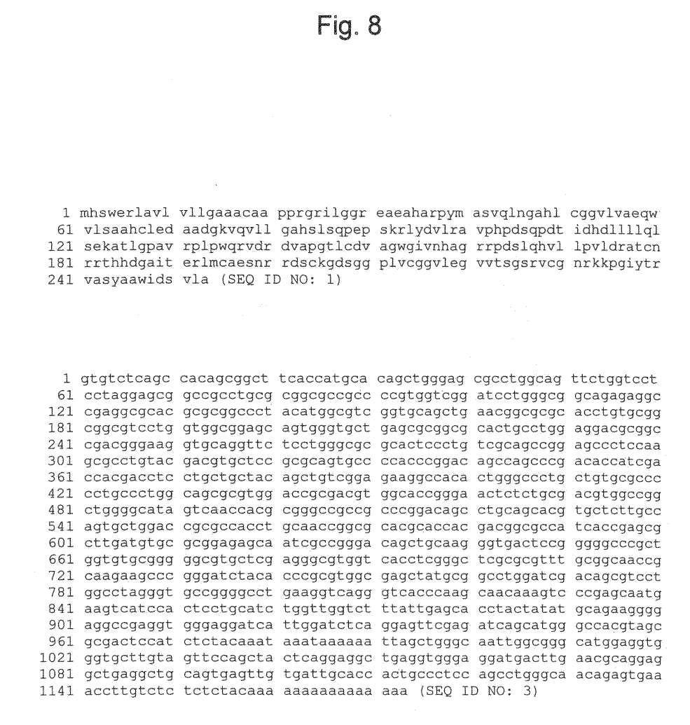

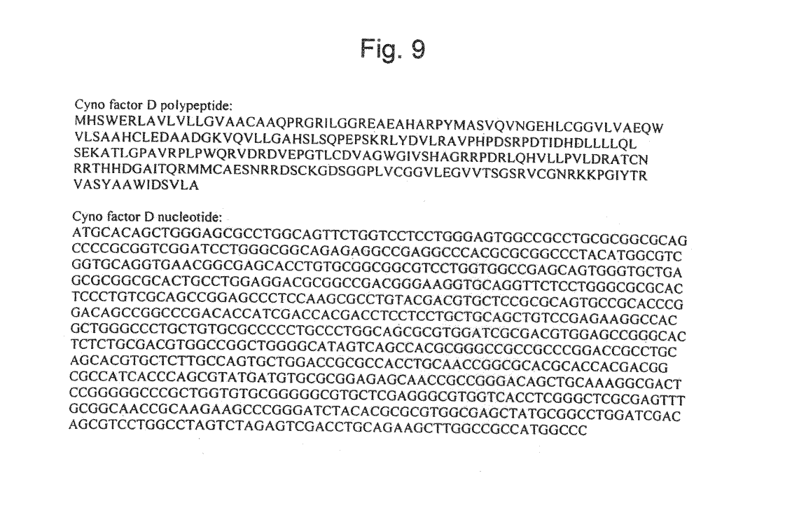

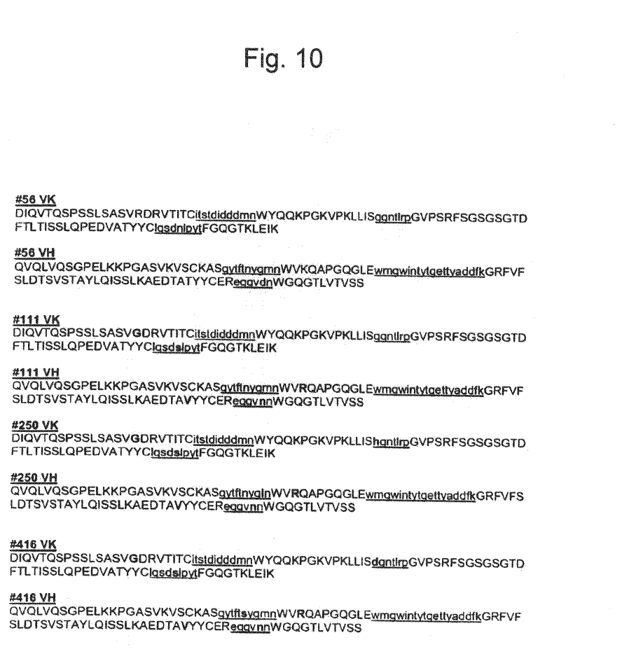

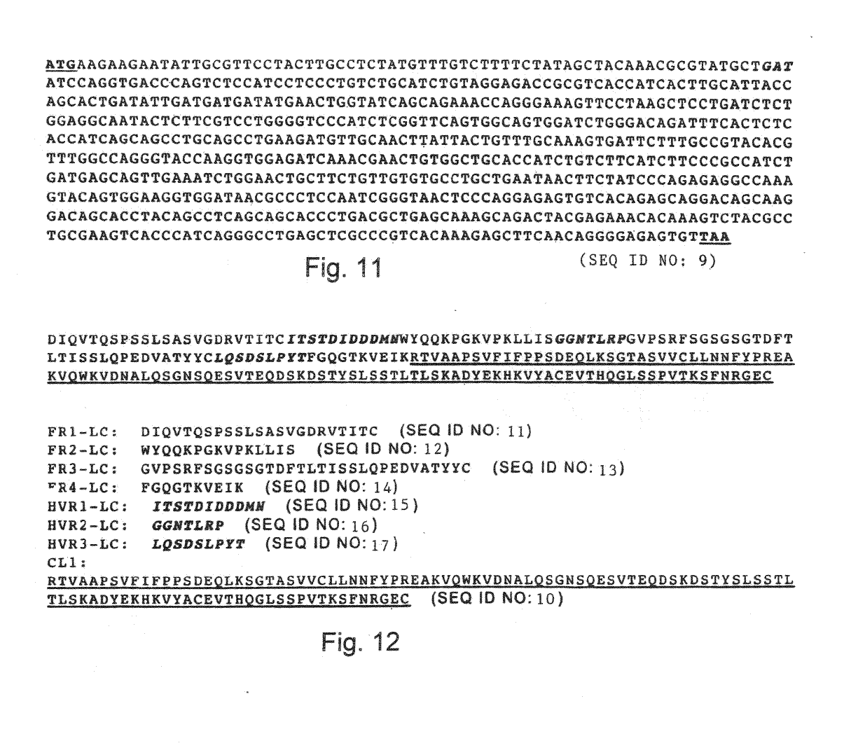

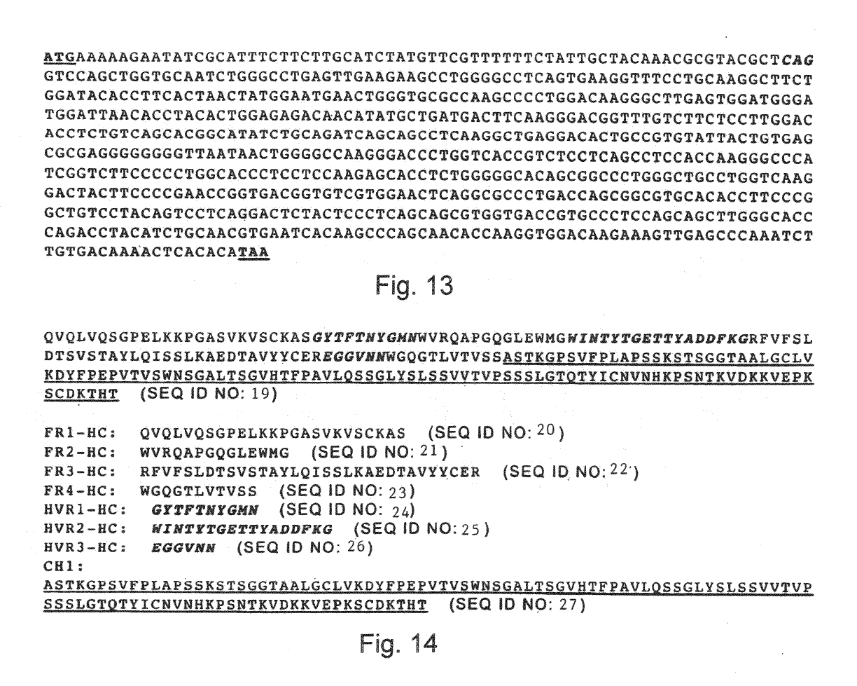

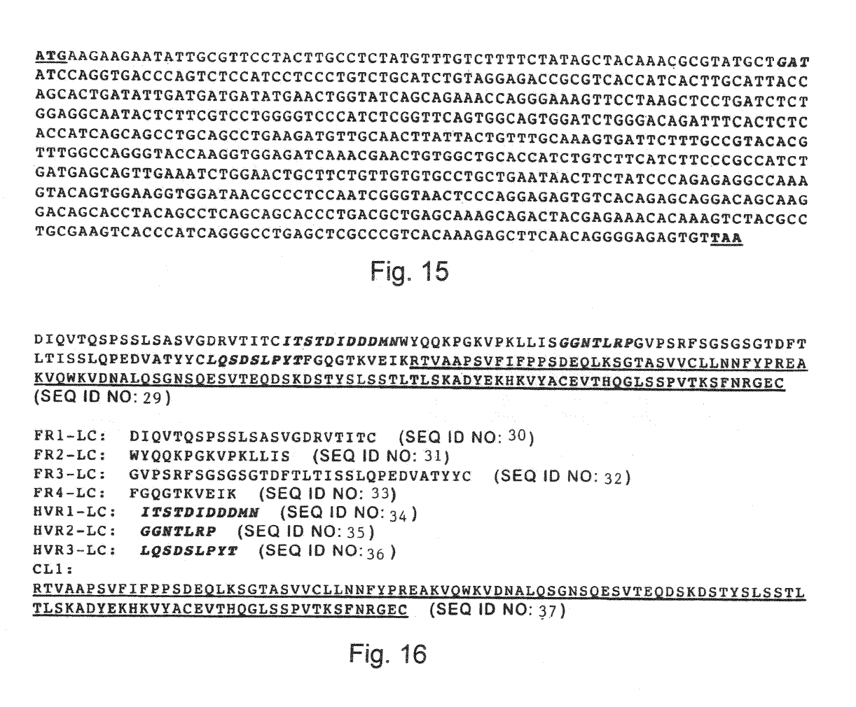

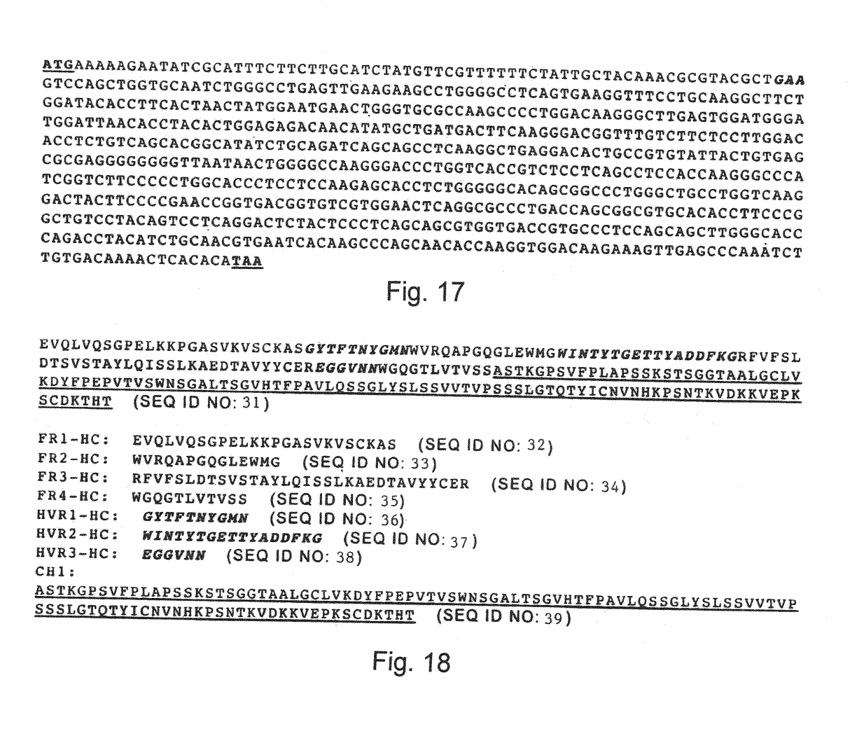

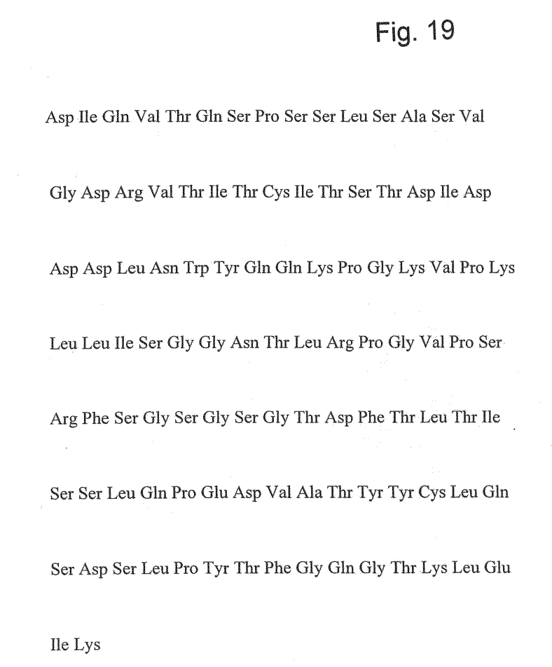

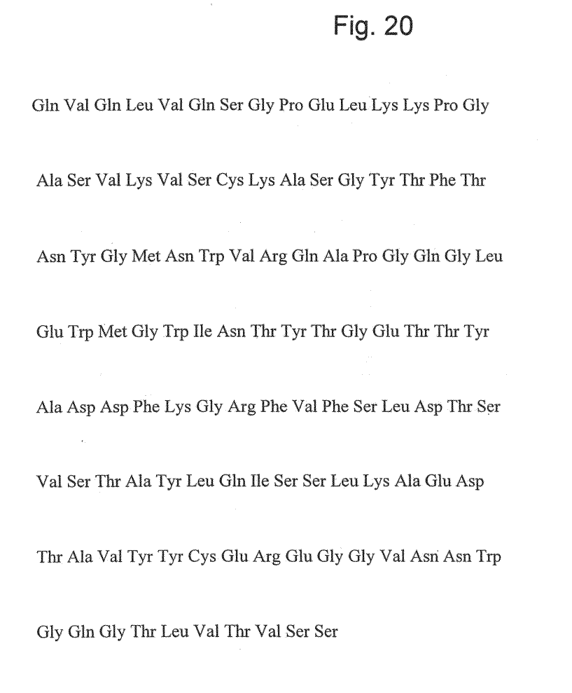

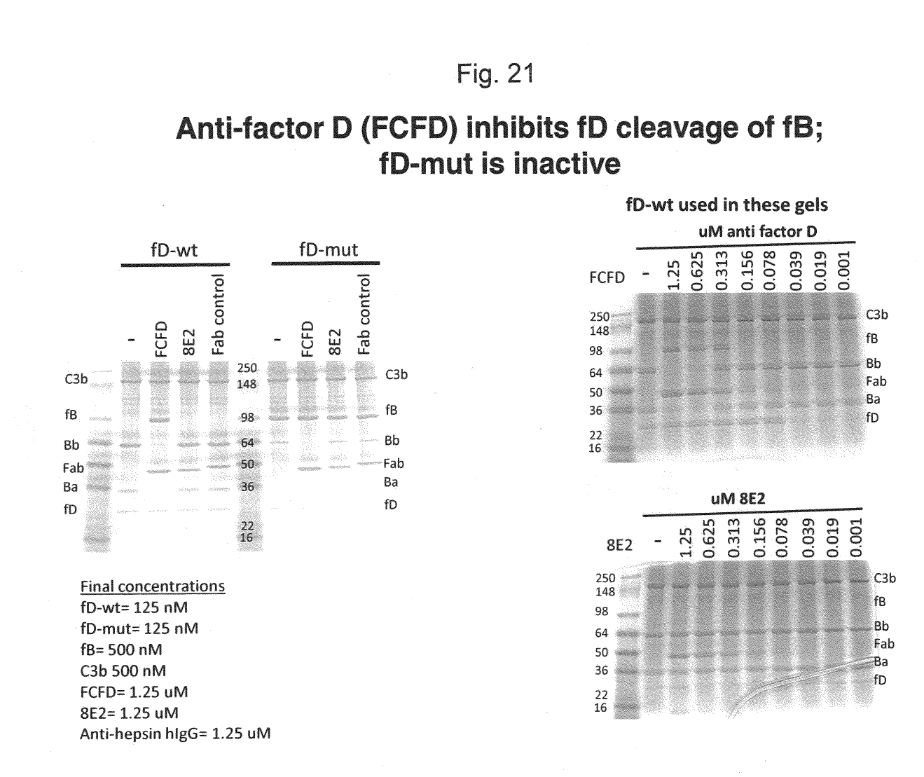

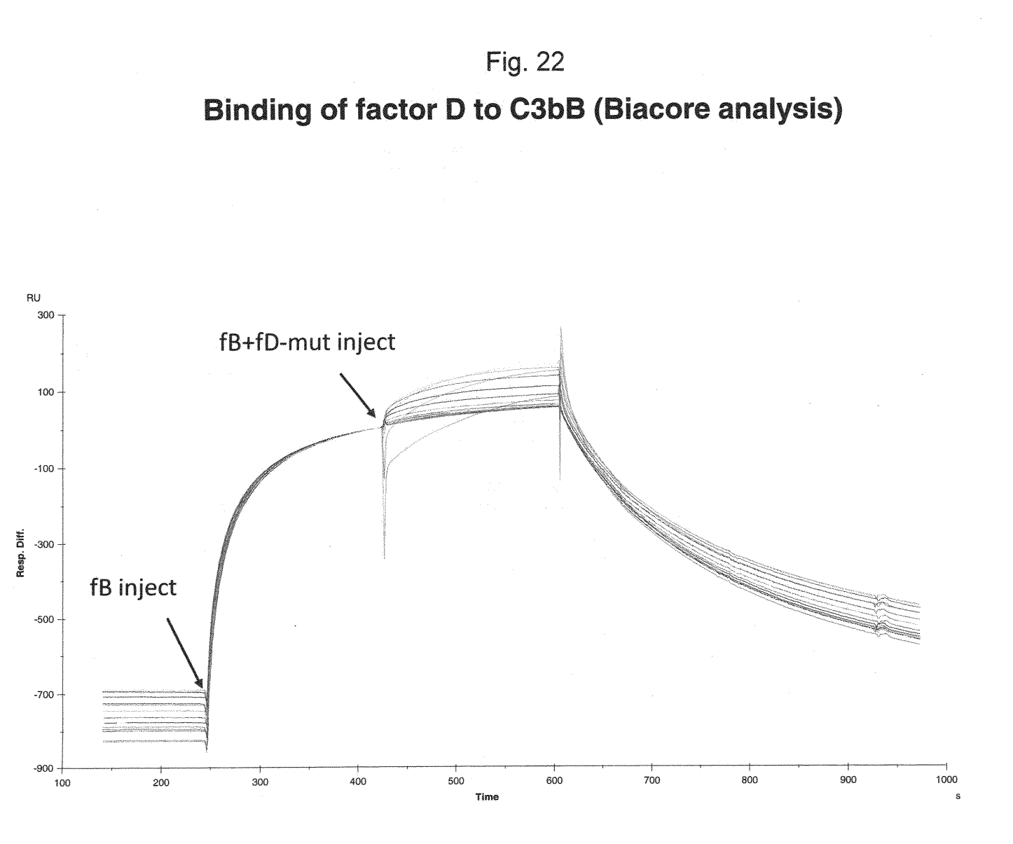

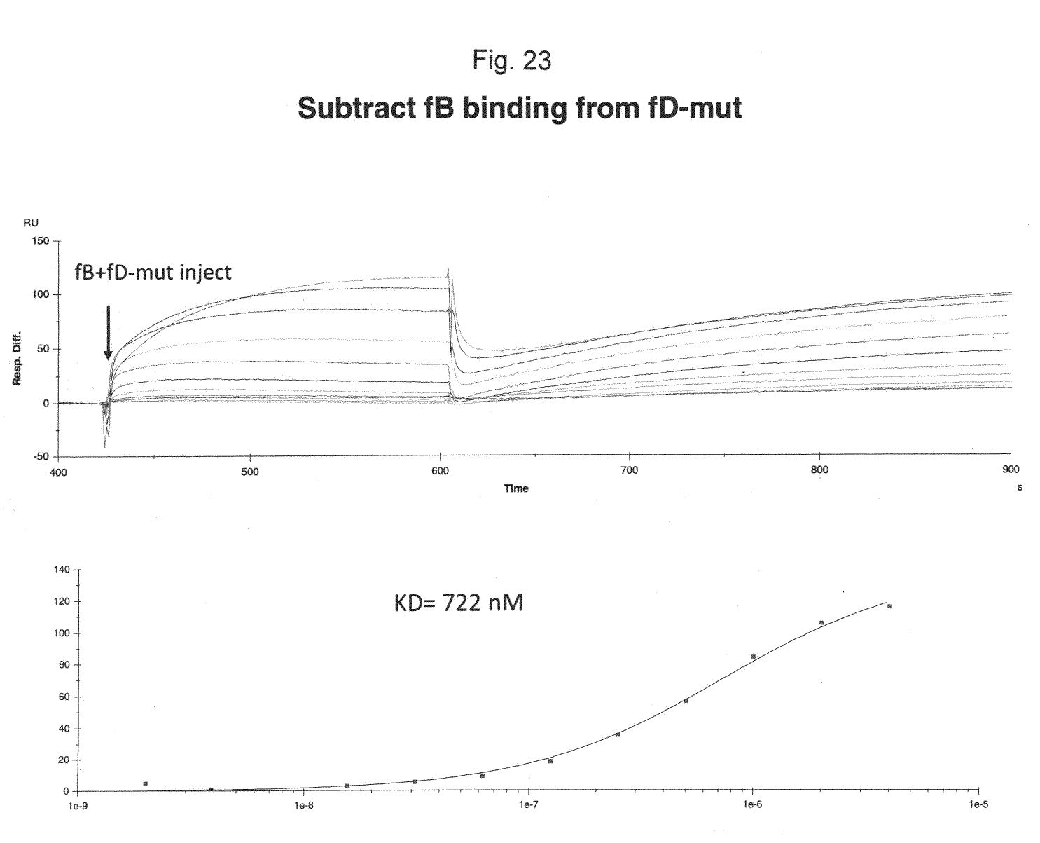

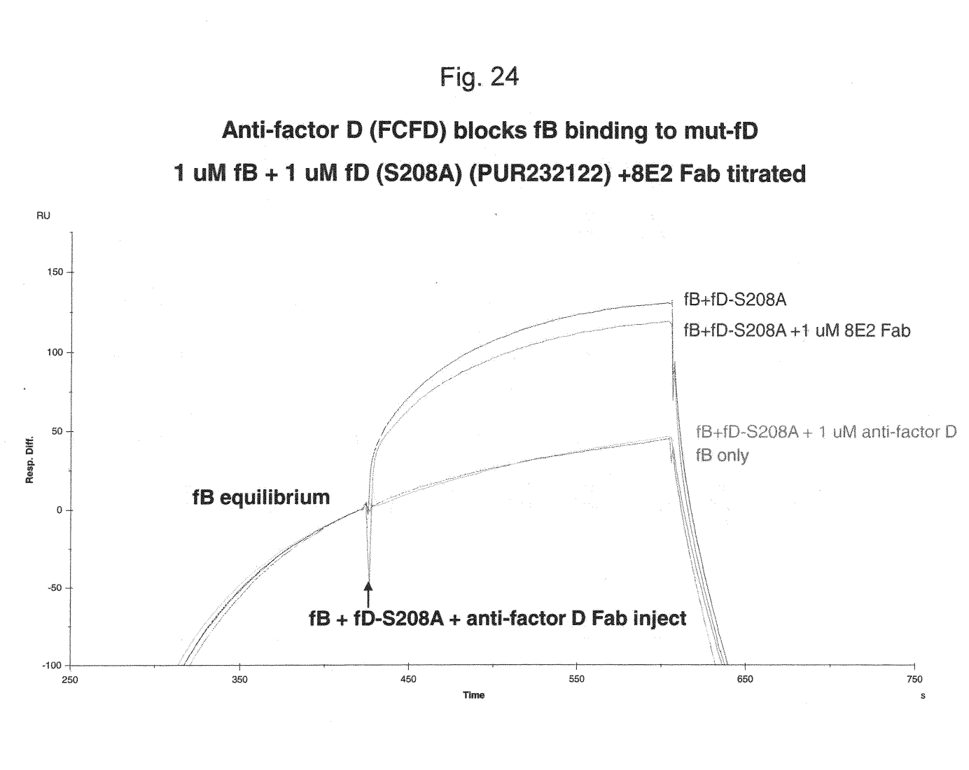

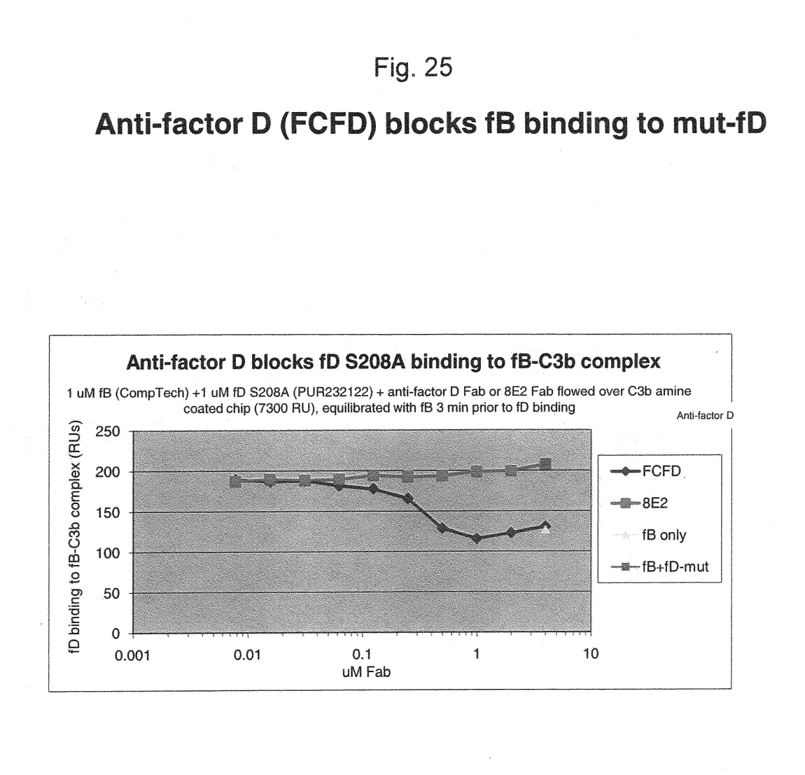

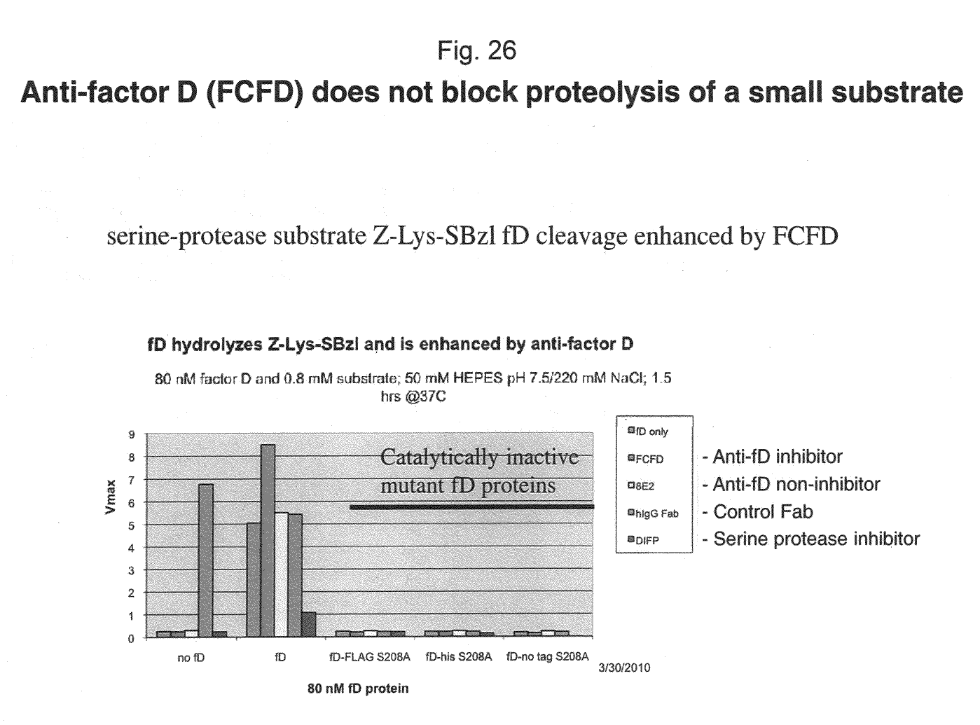

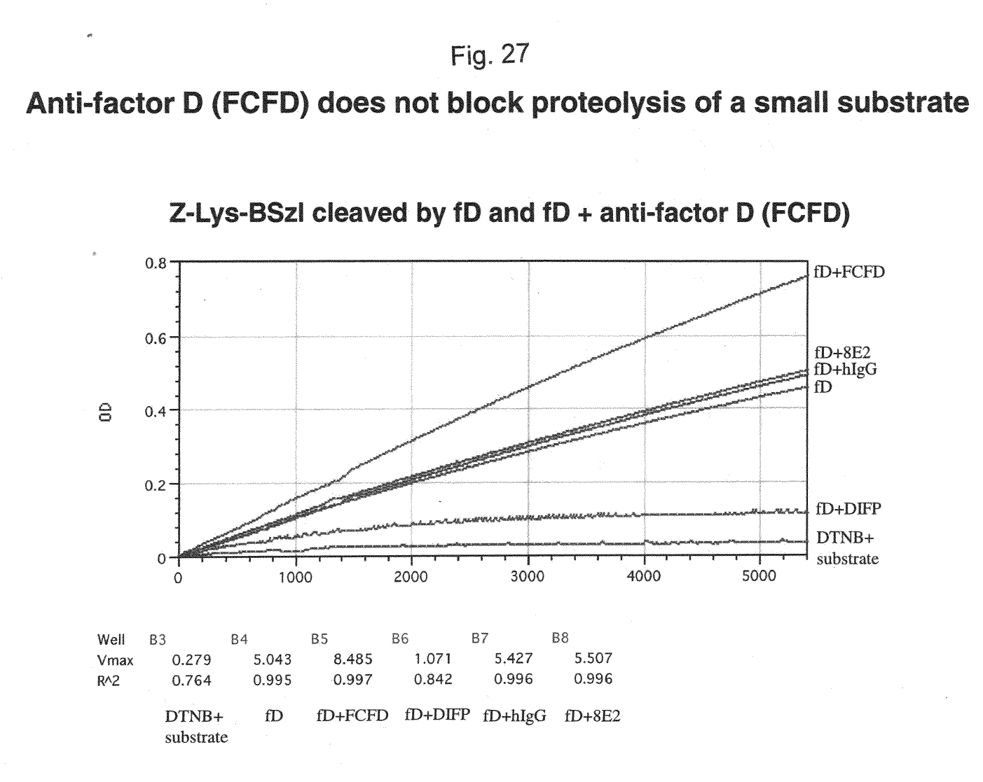

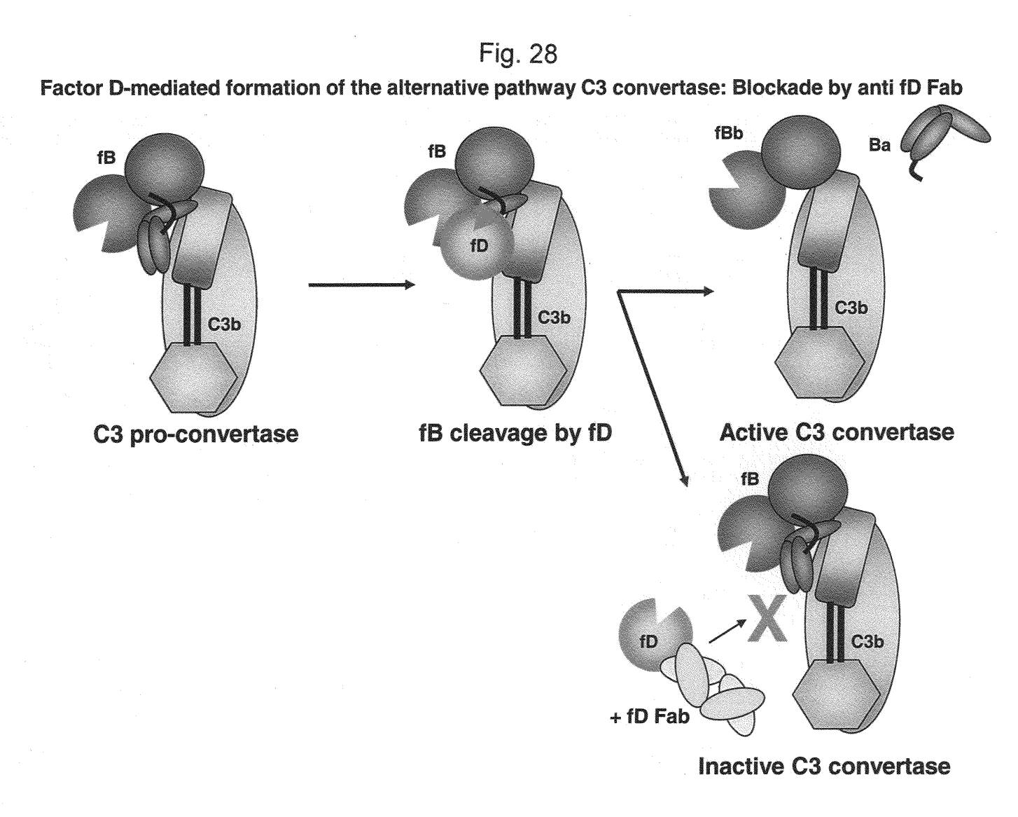

As shown in FIGS. 5, 6A and 6B, it has been determined that amino acid residues D131, V132, P134, D165, R166, A167, T168, N170, R171, R172, T173, D176, G177, I179, E181, R222, and K223 of human Factor D of SEQ ID NO: 1 participate in binding to an anti-factor B antibody Fab fragment. It has further been found that, for binding purposes, the key residue in the human Factor D amino acid sequence is R172, which can potentially form six or more hydrogen bonds with the anti-Factor D antibody heavy and light chains. FIGS. 6A and 6B also show the residues in the anti-Factor D antibody heavy and light chains which are in close proximity to and available to interact (for example by forming hydrogen bonds) with the human Factor D molecule.

Those of ordinary skill in the pertinent art will understand that a set of structure coordinates for a polypeptide complex is a relative set of points that define a shape in three dimensions. Therefore, it is possible that a different set of coordinates defines a similar or identical shape. Moreover, slight variations in the individual coordinates will have little effect on the overall shape.

In accordance with the present invention, the structure coordinates of a complex comprising Factor D and an anti-Factor D antibody or an antigen binding fragment thereof, for example a Fab fragment of an anti-Factor D monoclonal antibody, may be stored in a machine-readable storage medium, where the machine can be a computer. The data generated can be used for a variety of purposes, such as, for example, drug discovery, discovery of anti-Factor D antibody variants with improved properties, such as improved specific binding to Factor D, and X-ray crystallographic analysis of other protein crystals. In order to use the structure coordinates generated for the Factor D/anti-factor antibody complex, it is necessary to convert the structural coordinates into a three-dimensional shape. This can be readily accomplished through the use of commercially available software that is capable of generating a three-dimensional graphical representation of molecular complexes, or portions thereof, from a set of structure coordinates. Such three-dimensional representation is also within the scope of the present invention.

Thus, the invention includes a computer for producing a three-dimensional representation of: a molecular complex comprising a binding site defined by structure coordinates of amino acid residues D131, V132, P134, D165, R166, A167, T168, N170, R171, R172, T173, D176, G177, I179, E181, R222, and K223 of human Factor D of SEQ ID NO: 1, wherein the computer comprises: (i) a machine-readable data storage medium comprising a data storage material encoded with machine-readable data, wherein such data comprises the structure coordinates of amino acid residues D131, V132, P134, D165, R166, A167, T168, N170, R171, R172, T173, D176, G177, I179, E181, R222, and K223 of human Factor D of SEQ ID NO: 1, and (ii) instructions for processing the machine-readable data into said three-dimensional representation.

In certain embodiments, the computer comprises a display for displaying the structure coordinates.

In another aspect, the invention concerns a method for evaluating the potential of a chemical entity to associate with a molecular complex comprising a binding site defined by structure coordinates of amino acid residues D131, V132, P134, D165, R166, A167, T168, N170, R171, R172, T173, D176, G177, I179, E181, R222, and K223 of human Factor D of SEQ ID NO: 1, comprising the steps of: (i) employing computational means to perform a fitting operation between the chemical entity and said binding site of the molecular complex; and (ii) analyzing the results of the fitting operation to quantify the association between the chemical entity and said binding site. The chemical entity may, for example, be an agonist or antagonist of Factor D, including agonist and antagonist antibodies and variants of the anti-Factor D antibody used for determination of the crystal structure and three-dimensional confirmation of Factor D complexed with an anti-Factor D antibody herein, or an antigen binding fragment of such antibodies. The chemical entity may also be a peptide mimetic of an agonist or antagonist Factor D antibody or antibody fragment.

The potential agonist or antagonist may be synthesized, and contacted with Factor D to determine its ability to interact with (e.g. bind to) Factor D. It is further possible to determine whether a potential antagonist interrupts the Factor D/anti-Factor D antibody interaction. Before actually testing the binding of the potential antagonist to Factor D, it is possible, using the molecular coordinates and three-dimensional models provided by the present invention, to analyze the structure of such compound by computer modeling techniques. If computer modeling indicates a strong interaction or binding, the compound may then be produced (e.g. by synthetic and/or recombinant means) and tested for its ability to bind to Factor D.

Anti-Factor D Antibodies

Anti-Factor D antibodies are selected using a Factor D antigen derived from a mammalian species. Preferably the antigen is human Factor D. However, Factor Ds from other species such as cyno or murine Factor D can also be used as the target antigen. The Factor D antigens from various mammalian species may be isolated from natural sources. In other embodiments, the antigen is produced recombinantly or made using other synthetic methods known in the art.

The antibody selected following the methods of the present invention will normally have a sufficiently strong binding affinity for the Factor D antigen. For example, the antibody may bind human Factor D with a K_{d} value of no more than about 5 nM, preferably no more than about 2 nM, and more preferably no more than about 500 pM. Antibody affinities may be determined by a surface plasmon resonance based assay (such as the BIAcore assay as described in Examples); enzyme-linked immunoabsorbent assay (ELISA); and competition assays (e.g. RIA's), for example.

Also, the antibody may be subject to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and in vitro and in vivo assays described below for identifying Factor D antagonists.

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al. (1995) J. Biol. Chem. 270:1388-1394, can be performed to determine whether the antibody binds an epitope of interest.

In a preferred embodiment, the anti-Factor D antibodies are selected using a unique phage display approach. The approach involves generation of synthetic antibody phage libraries based on single framework template, design of sufficient diversities within variable domains, display of polypeptides having the diversified variable domains, selection of candidate antibodies with high affinity to target Factor D antigen, and isolation of the selected antibodies.

Details of the phage display methods can be found, for example, in WO90/05144; WO90/14424; WO90/14430, WO92/01047, WO93/11236; WO91/05058; WO03/102157; WO91/05058; U.S. Pat. No. 6,291,158; U.S. Pat. No. 6,291,159, U.S. Pat. No. 6,291,160, U.S. Pat. No. 6,291,161; U.S. Pat. No. 5,969,108, U.S. Pat. No. 5,885,793; and U.S. Pat. No. 5,643,768.

Factor D antibodies are disclosed in US Patent Publication Nos. 20020081293, 20080118506, 20090181017, and 20090269338, the entire disclosures of which are hereby expressly incorporated by reference.

Preferred antibodies include antibody clones #56, #111, #250, and #416, the variable heavy chain and the variable light chain amino acid sequences of which are shown in FIG. 10 (SEQ ID NOs: 5, 6, 7 and 8, respectively).

Further preferred anti-Factor D antibody is anti-Factor D Fab 238. The nucleotide sequence (SEQ ID NO: 9) of the light chain of humanized anti-Factor D Fab 238 is shown in FIG. 11 (SEQ ID NO: 9). The nucleotide sequence encodes for the light chain of humanized anti-Factor D Fab 238 with the start and stop codons shown in bold and underlined. The codon corresponding to the first amino acid in FIG. 11 (SEQ ID NO: 10) is bold and italicized. FIG. 12 shows the amino acid sequence (SEQ ID NO: 10) of the light chain for humanized anti-Factor D Fab 238. The amino acid sequence lacks the N-terminus signal sequence of the polypeptide encoded by SEQ ID NO: 9 shown in FIG. 11. The HVR sequences are bold and italicized. Variable regions are regions not underlined while first constant domain CL1 is underlined. Framework (FR) regions and HVR regions are shown. FIG. 13 shows the nucleotide sequence (SEQ ID NO: 18) of the heavy chain of humanized anti-Factor D Fab 238. The nucleotide sequence encodes for the heavy chain of humanized anti-Factor D Fab 238 with the start and stop codon shown in bold and underlined. The codon corresponding to the first amino acid in FIG. 14 (SEQ ID NO: 19) is bold and italicized. FIG. 14 shows the amino acid sequence (SEQ ID NO: 19) of the heavy chain for humanized anti-Factor D Fab 238. The amino acid sequence lacks the N-terminus signal sequence of the polypeptide encoded by SEQ ID NO: 18 shown in FIG. 13. The HVR sequences are bold and italicized. Variable regions are regions not underlined while first constant domain CH1 is underlined. Framework (FR) regions and HVR regions are shown.

A still further preferred anti-Factor D antibody is anti-Factor D Fab 238-1. FIG. 15 shows the nucleotide sequence (SEQ ID NO: 28) of the light chain of humanized anti-Factor D Fab 238-1. The nucleotide sequence encodes for the light chain of humanized anti-Factor D Fab 238-1 with the start and stop codon shown in bold and underlined. The codon corresponding to the first amino acid in FIG. 16 (SEQ ID NO: 29) is bold and italicized. FIG. 16 shows the amino acid sequence (SEQ ID NO 29) of the light chain for humanized anti-Factor D Fab 238-1. The amino acid sequence lacks the N-terminus signal sequence of the polypeptide encoded by SEQ ID NO: 28 shown in FIG. 15. The HVR sequences are bold and italicized. Variable regions are regions not underlined while first constant domain CL1 is underlined. Framework (FR) regions and HVR regions are shown. FIG. 17 shows the nucleotide sequence (SEQ ID NO: 30) of the heavy chain of humanized anti-Factor D Fab 238-1. The nucleotide sequence encodes for the heavy chain of humanized anti-Factor D Fab 238-1 with the start and stop codon in bold and underlined. The codon corresponding to the first amino acid in FIG. 18 (SEQ ID NO: 31) is bold and italicized. FIG. 18 shows the amino acid sequence (SEQ ID NO: 31) of the heavy chain for humanized anti-Factor D Fab 238-1. The amino acid sequence lacks the N-terminus signal sequence of the polypeptide encoded by SEQ ID NO: 30 shown in FIG. 18. The HVR sequences are bold and italicized. Variable regions are regions not underlined while first constant domain CH1 is underlined. Framework (FR) regions and HVR regions are shown.

Another preferred anti-Factor D antibody is anti-factor D Fab 238-2, having the light chain amino acid sequence shown in FIG. 19 (SEQ ID NO: 40) and the heavy chain amino acid sequence shown in FIG. 20 (SEQ ID NO: 41).

Preferred mimetics, e.g. peptidomimetics, mimic the binding and/or biological properties of the preferred antibodies or antibody fragments herein.

Uses of Factor D Antibodies and Other Factor D Antagonists

The invention herein provides Factor D antagonists, including anti-Factor D antibodies, and variants thereof, and fragments thereof (e.g. antigen-binding fragments) useful for the prevention and treatment of complement-associated conditions, including eye conditions (all eye conditions and diseases the pathology of which involves complement, including the classical and the alternative pathways, and in particular the alternative pathway of complement), such as, for example, macular degenerative diseases, such as all stages of age-related macular degeneration (AMD), including dry and wet (non-exudative and exudative) forms, choroidal neovascularization (CNV), uveitis, diabetic and other ischemia-related retinopathies, endophthalmitis, and other intraocular neovascular diseases, such as diabetic macular edema, pathological myopia, von Hippel-Lindau disease, histoplasmosis of the eye, Central Retinal Vein Occlusion (CRVO), corneal neovascularization, and retinal neovascularization. One group of complement-associated eye conditions includes age-related macular degeneration (AMD), including non-exudative (e.g. intermediate dry AMD or geographic atrophy (GA)) and exudative (e.g. wet AMD (choroidal neovascularization. (CNV)) AMD, diabetic retinopathy (DR), endophthalmitis and uveitis. In one example, complement-associated eye condition is intermediate dry AMD. In one example, complement-associated eye condition is geographic atrophy. In one example, complement-associated eye condition is wet AMD (choroidal neovascularization (CNV)).

AMD is age-related degeneration of the macula, which is the leading cause of irreversible visual dysfunction in individuals over the age of 60. Two types of AMD exist, non-exudative (dry) and exudative (wet) AMD. The dry, or nonexudative, form involves atrophic and hypertrophic changes in the retinal pigment epithelium (RPE) underlying the central retina (macula) as well as deposits (drusen) on the RPE. Patients with nonexudative AMD can progress to the wet, or exudative, form of AMD, in which abnormal blood vessels called choroidal neovascular membranes (CNVMs) develop under the retina, leak fluid and blood, and ultimately cause a blinding disciform scar in and under the retina. Nonexudative AMD, which is usually a precursor of exudative AMD, is more common. The presentation of nonexudative AMD varies: hard drusen, soft drusen, RPE geographic atrophy, and pigment clumping can be present. Complement components are deposited on the RPE early in AMD and are major constituents of drusen.

Factor D antagonists can be evaluated in a variety of cell-based assays and animal models of complement-associated diseases or disorders.

Thus, for example, recombinant (transgenic) animal models can be engineered by introducing the coding portion of the genes of interest into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g. baboons, chimpanzees and other monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA 82, 6148-615 [1985]); gene targeting in embryonic stem cells (Thompson et al., Cell 56, 313-321 [1989]); electroporation of embryos (Lo, Mol. Cell. Biol. 3, 1803-1814 [1983]); sperm-mediated gene transfer (Lavitrano et al., Cell 57, 717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA 89, 623-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry.

The animals may be further examined for signs of immune disease pathology, for example by histological examination to determine infiltration of immune cells into specific tissues. Blocking experiments can also be performed in which the transgenic animals are treated with a candidate Factor D antagonist to determine the extent of effects on complement and complement activation, including the classical and alternative pathways, or T cell proliferation. In these experiments, blocking antibodies which bind to the polypeptide of the invention, are administered to the animal and the biological effect of interest is monitored.

Alternatively, “knock out” animals can be constructed which have a defective or altered gene encoding Factor D, as a result of homologous recombination between the endogenous gene encoding the Factor D polypeptide and altered genomic DNA encoding the same polypeptide introduced into an embryonic cell of the animal. For example, cDNA encoding Factor D can be used to clone genomic DNA encoding Factor D in accordance with established techniques. A portion of the genomic DNA encoding Factor D can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the Factor D polypeptide.

Thus, the biological activity of potential Factor D antagonists can be further studied in murine Factor D knock-out mice.

An animal model of age-related macular degeneration (AMD) consists of mice with a null mutation in Ccl-2 or Ccr-2 gnes. These mice develop cardinal features of AMD, including accumulation of lipofuscin in and drusen beneath the retinal pigmented epithelium (RPE), photoreceptor atrophy and choroidal neovascularization (CNV). These features develop beyond 6 months of age. Candidate Factor D antagonists can be tested for the formation of drusen, photoreceptor atrophy and choroidal neovascularization.

Pharmaceutical Compositions

Therapeutic formulations of the polypeptide or antibody, or antibody fragment thereof (e.g. antigen-binding fragment), or antibody variant thereof, may be prepared for storage as lyophilized formulations or aqueous solutions by mixing the polypeptide having the desired degree of purity with optional. “pharmaceutically-acceptable” carriers, excipients or stabilizers typically employed in the art (all of which are termed “excipients”). For example, buffering agents, stabilizing agents, preservatives, isotonifiers, non-ionic detergents, antioxidants and other miscellaneous additives. (See. Remington's Pharmaceutical Sciences, 16th edition, A. Osol, Ed. (1980)). Such additives must be nontoxic to the recipients at the dosages and concentrations employed.

Buffering agents help to maintain the pH in the range which approximates physiological conditions. They are preferably present at concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers. (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, there may be mentioned phosphate buffers, histidine buffers and trimethylamine salts such as Tris.

Preservatives may be added to retard microbial growth, and may be added in amounts ranging from 0.2%-1% (w/v). Suitable preservatives for use with the present invention include phenol, benzyl alcohol, meta-cresol, methyl paraben, propyl paraben, octadecyldimethylbenzyl ammonium chloride, benzalconium halides (e.g., chloride, bromide, iodide), hexamethonium chloride, alkyl parabens such as methyl or propyl paraben, catechol, resorcinol, cyclohexanol, and 3-pentanol.

Isotonicifiers sometimes known as “stabilizers” may be added to ensure isotonicity of liquid compositions of the present invention and include polhydric sugar alcohols, preferably trihydric or higher sugar alcohols, such as glycerin, erythritol, arabitol, xylitol, sorbitol and mannitol.

Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, .alpha.-monothioglycerol and sodium thio sulfate; low molecular weight polypeptides (i.e. <10 residues); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; polysaccharides such as dextran. Stabilizers may be present in the range from 0.1 to 10,000 weights per part of weight active protein.

Non-ionic surfactants or detergents (also known as “wetting agents”) may be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic® polyols, polyoxyethylene sorbitan monoethers (Tween®-20, Tween®-80, etc.). Non-ionic surfactants may be present in a range of about 0.05 mg/ml to about 1.0 mg/ml, preferably about 0.07 mg/ml to about 0.2 mg/ml.

Additional miscellaneous excipients include bulking agents, (e.g. starch), chelating agents (e.g. EDTA), antioxidants (e.g., ascorbic acid, methionine, vitamin E), and cosolvents. The formulation herein may also contain more than one active compound as necessary for the particular indication being treated, preferably those with complementary activities that do not adversely affect each other. For example, it may be desirable to further provide an immunosuppressive agent. Such molecules are suitably present in combination in amounts that are effective for the purpose intended. The active ingredients may also be entrapped in microcapsule prepared, for example, by coascervation techniques or by interfacial polymerization, for example, hydroxymethylcellulose or gelatin-microcapsule and poly-(methylmethacylate) microcapsule, respectively, in colloidal drug delivery systems (for example, liposomes, albumin micropheres, microemulsions, nano-particles and nanocapsules) or in macroemulsions. Such techniques are disclosed in Remington's Pharmaceutical Sciences, 16th edition, A. Osal, Ed. (1980).

The formulations to be used for in vivo administration must be sterile. This is readily accomplished, for example, by filtration through sterile filtration membranes. Sustained-release preparations may be prepared. Suitable examples of sustained-release preparations include semi-permeable matrices of solid hydrophobic polymers containing the antibody, or antibody variant or fragment (e.g. antigen-binding fragment) thereof, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOTT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C. resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The compounds that can be identified by the method of the present invention for prevention or treatment of an ocular disease or condition are typically administered by ocular, intraocular, and/or intravitreal injection, and/or juxtascleral injection, and/or subtenon injection, and/or superchoroidal injection and/or topical administration in the form of eyedrops and/or ointment. Such compounds of the invention may be delivered by a variety of methods, e.g. intravitreally as a device and/or a depot that allows for slow release of the compound into the vitreous, including those described in references such as Intraocular Drug Delivery, Jaffe, Jaffe, Ashton, and Pearson, editors, Taylor & Francis (March 2006). In one example, a device may be in the form of a minpump and/or a matrix and/or a passive diffusion system and/or encapsulated cells that release the compound for a prolonged period of time (Intraocular Drug Delivery, Jaffe, Jaffe, Ashton, and Pearson, editors, Taylor & Francis (March 2006). Other methods of administration may also be used, which includes but is not limited to, topical, parenteral, subcutaneous, intraperitoneal, intrapulmonary, intranasal, and intralesional administration. Parenteral infusions include intramuscular, intravenous, intraarterial, intraperitoneal, or subcutaneous administration.

Formulations for ocular, intraocular or intravitreal administration can be prepared by methods and using ingredients known in the art. A main requirement for efficient treatment is proper penetration through the eye. Unlike diseases of the front of the eye, where drugs can be delivered topically, retinal diseases require a more site-specific approach. Eye drops and ointments rarely penetrate the back of the eye, and the blood-ocular barrier hinders penetration of systemically administered drugs into ocular tissue. Accordingly, usually the method of choice for drug delivery to treat retinal disease, such as AMD and CNV, is direct intravitreal injection. Intravitrial injections are usually repeated at intervals which depend on the patient's condition, and the properties and half-life of the drug delivered. For intraocular (e.g. intravitreal) penetration, usually molecules of smaller size are preferred.

The efficacy of the treatment of complement-associated eye conditions, such as AMD or CNV, can be measured by various endpoints commonly used in evaluating intraocular diseases. For example, vision loss can be assessed. Vision loss can be evaluated by, but not limited to, e.g., measuring by the mean change in best correction visual acuity (BCVA) from baseline to a desired time point (e.g., where the BCVA is based on Early Treatment Diabetic Retinopathy Study (ETDRS) visual acuity chart and assessment at a test distance of 4 meters), measuring the proportion of subjects who lose fewer than 15 letters in visual acuity at a desired time point compared to baseline, measuring the proportion of subjects who gain greater than or equal to 15 letters in visual acuity at a desired time point compared to baseline, measuring the proportion of subjects with a visual-acuity Snellen equivalent of 20/2000 or worse at a desired time point, measuring the NEI Visual Functioning Questionnaire, measuring the size of CNV and amount of leakage of CNV at a desired time point, e.g., by fluorescein angiography, etc. Ocular assessments can be done, e.g., which include, but are not limited to, e.g., performing eye exam, measuring intraocular pressure, assessing visual acuity, measuring slitlamp pressure, assessing intraocular inflammation, etc.

The amount of therapeutic polypeptide, antibody, or antibody variant thereof, or fragment thereof (e.g antigen-binding fragment) which will be effective in the treatment of a particular disorder or condition will depend on the nature of the disorder or condition, and can be determined by standard clinical techniques. Where possible, it is desirable to determine the dose-response curve and the pharmaceutical compositions of the invention first in vitro, and then in useful animal model systems prior to testing in humans.

In one embodiment, an aqueous solution of therapeutic polypeptide, antibody, or antibody variant thereof, or fragment thereof (e.g. antigen-binding fragment), is administered by subcutaneous injection. In another embodiment, an aqueous solution of therapeutic polypeptide, antibody, or antibody variant thereof, or fragment thereof (e.g. antigen-binding fragment) is administered by intravitreal injection. Each dose may range from about 0.5.mu.g to about 50.mu.g per kilogram of body weight, or more preferably, from about 3.mu.g to about 30.mu.g per kilogram body weight.

The dosing schedule for subcutaneous administration may vary form once a month to daily depending on a number of clinical factors, including the type of disease, severity of disease, and the subject's sensitivity to the therapeutic agent.

The following examples are offered for illustrative purposes only, and are not intended to limit the scope of the present invention in any way.

All patent and literature references cited in the present specification are hereby expressly incorporated by reference in their entirety.

### Example 1

Determination of Co-Crystal Structure of Factor D and Anti-Factor D Antibody

Both human and cyno factor D were expressed in Chinese Hamster Ovary (CHO) cells. Purification was performed by passing the CHO cell supernatant over an anti-Factor D affinity column. Proteins were eluted with 0.1 M acidic and with 4% v/v 1.5 M Tris pH 8.6 and dialyzed in a buffer containing 10 mM Hepes pH 7.2 with 140 mM NaCl. Anti-factor D Fab were provided in lyophilized formulation and reconstituted with water. Resulting solution is 50 mg/mL protein in 40 mM Histidine Hydrochloride, 20 mM Sodium Chloride, 180 mM Sucrose, 0.04% (w/v) polysorbate 20, pH 5.4.

Human factor D protein and anti-factor D Fab were mixed 1:1 ratio and purified over a Superdex 200 column pre-equilibrated with 20 mM hepes, pH 7.2 and 200 mM NaCl. The peak fractions containing the complex were pooled, concentrated to 30 mg/ml and used in crystallization trial. Crystals were grown at 4° C. using vapor diffusion method in sitting drops. Crystallization buffer containing 0.1 M TrisCl, pH 8.5, 0.2 M ammonium phosphate, 50% MPD and 0.01 M hexamine cobalt (III) chloride was mixed in equal volume with protein solution. Crystals appeared after 6 days and belonged to space group P4₃2₁2. The crystals were flash frozen in liquid nitrogen. A 2.4 Å data set was collected at SSRL Synchrotron Source on beam line 9-2.

The cyno factor D/anti-factor D Fab complex was purified following the same protocol as described above. Crystals used in the structure determination were grown at 19° C. from the following condition: 0.1 M MES pH 6.5, 25% PEG 550 MME, 0.01 M zinc sulfate and 3% 6-aminohexanoic acid using vapor diffusion method in sitting drops containing equal volume of protein solution (20 mg/ml) and mother liquor. The crystals appeared after 1 day and belonged to space group C2 with cell dimensions of a=132.048, b=132.048; c=180.288 Å for human Factor D:Fab complex, and a=182.205; b=80.673; c=142.575 Å for cyno factor D:Fab complex. The crystals were dipped in artificial mother liquor containing 10% glycerol and flash frozen in liquid nitrogen. A 2.1 Å data set was collected at an SSRL Synchrotron Source on beam line 9-2.

### Example 2

Preparation of Catalytically Inactive Factor D Protein

Full-length factor D cDNA was cloned into a pRK expression vector. Residue S208 (from starting methionine; S195 using trypsin numbering), which is part of the catalytic triad, was mutated to an alanine using QuickChange XL Site Directed Mutagenesis kit following manufactures instructions (Stratagene (Agilent), Santa Clara, Calif.). Protein was expressed in CHO cells and purified by passing supernatant multiple times over an Affi-Gel 10 (Bio-Rad) coupled with 26 mg/ml anti-Factor D antibody and eluted at pH 3.0. Proteins were further purified using a secondary 10/10 MonoS pH 6.0 column, concentrated with an Amicon Ultra-10 kD centrifugation filter (Millipore, Billerica, Mass.) and dialyzed in PBS. Protein sequence was verified with N-terminal sequencing using MALDI mass spectrometry.

### Example 3

Blocking of Factor B Cleavage by Anti-Factor D Antibody in a Fluid Phase Alternative Pathway C3 Convertase Assay

The assay buffer was 0.1% gelatin veronal buffer/10 mM MgCl₂, final concentration of components were 0.125 μM Factor D, 0.5 μM factor B, 0.5 μM C3b and 5 μM Fab antibodies (anti-Factor D, 8E2, control human Fab). 10 μl Factor D (0.5 μM) and 10 μl Fab (20 μM) were mixed for 15 min. 10 μl Factor B (2 μM) and 10 μl C3b (2 μM) were added to Factor D-Fab mixture and incubated for 30 minutes at 37 C. 40 μl Lammeli's buffer was added to stop the reaction. Samples were boiled for 5 minutes and run on a Novex 4-20% Tris-glycine polyacrylamide gel for 1.5 hours at 125 mV (SeeBlue2 MW marker). Gels were stained for one hour with SimplyBlue SafeStain, washed overnight with double distilled water and dried between Cellophane. As shown in FIG. 21, factor B cleavage was blocked by the anti-Factor D antibody but not by 8E2. The results show that the anti-factor D antibody blocks cleavage of factor B in a fluid phase alternative pathway C3 convertase assay.

### Example 4

Factor D (S208A) Binds Pro-Convertase with an Affinity of 772 nM (Bioacore Analysis)

Binding analysis was performed on Biacore 3000. C3b was amine coupled to CM5 chip following manufacturer's recommendation. The CM5 chip was activated with N-hydroxyl succinimide and N-ethyl-N′-(dimethylaminopropyl)-carbodiimide, flow 5 μl/min, 30 μl. C3b (50 μg/ml) was flowed for 5 μl/min, 20 μl to achieve 7300 RU final. Factor B, Factor D, anti-Factor D antibody and 8E2 Fab fragment proteins and antibodies were buffer exchanged using GE Healthcare's AKTA in assay buffer: veronal buffer/1 mM NiCl₂/0.05% Surfactant P-20. Binding assays used “Coinject” program. One μM factor B was injected (flow 30 μl/minute, 90 μl) followed by coinject mix of 1 μM factor B and Factor D dilutions (flow 30 μl/minute, 90 μl) then allowed to dissociate in assay buffer for 5 minutes. Chip was regenerated with three, one minute washes with 3 M NaCl in 50 mM sodium acetate pH 5.0 and washed 5 minutes with buffer. As shown in FIGS. 22 and 23, Factor (S208A) binds to C3bB pro-convertase with an affinity of 772 nM as determined by this Biacore analysis.

### Example 5

Anti-Factor D Antibody Blocks Factor D Binding

Binding analysis was performed on Biacore 3000. C3b was amine coupled to CM5 chip following manufacturer's recommendation. The CM5 chip was activated with N-hydroxyl succinimide and N-ethyl-N′-(dimethylaminopropyl)-carbodiimide, flow 5 μl/min, 30 μl. C3b (50 μg/ml) was flowed for 5 μl/min, 20 μl to achieve 6020 RU final. Factor B, factor D, anti-Factor D antibody and 8E2 Fab fragment proteins and antibodies were buffer exchanged using GE Healthcare's AKTA in assay buffer: veronal buffer/1 mM NiCl₂/0.05% Surfactant P-20. Binding assays used “Coinject” program. One μM factor B was injected (flow 30 μl/minute, 90 μl) followed by coinject mix of 1 μM factor B, 1 μM Factor D and Fab antibody dilutions (flow 30 μl/minute, 90 μl) then allowed to dissociate in assay buffer for 5 minutes. Chip was regenerated with three, one-minute washes with 3 M NaCl in 50 mM sodium acetate pH 5.0 and washed 5 minutes with buffer. As shown in FIGS. 24 and 25, the anti-Factor D antibody blocked Factor D (S208A) binding to C3bB pro-convertase.

### Example 6

Anti-Factor D Antibody does not Affect Catalytic Cleavage

Small substrates are efficiently cleaved by Factor D bound to anti-Factor D antibody confirming the absence of major conformational changes in the Factor D active site. Factor D hydrolysis of thioester benzyl substrate Z-Lys-SBzl was measured using DTNB (Ellman's reagent (5,5′-dithiobis-(2-nitrobenzoic acid)) in assay buffer 50 mM HEPES pH7.5/220 mM NaCl. Stock solutions of 3.2 mM Z-lys-SBzl in DMSO (dimethyl sulfoxide), 320 nM Factor D proteins (Genentech), 3.2 μM antibodies and 8 mM DTNB in assay buffer, and 200 mM DIFP (diisopropyl fluorophosphates) in isopropanol were made. Assay volume was 200 μl in assay buffer with final concentrations of 2 mM DTNB, 800 nM Z-Lys-SBzl, 80 nM Factor D, 800 uM antibodies or 20 mM DIFP. Hydrolosis rates were measured in a Spectramax Plus 384 spectrophotometer at 450 nm for 1.5 hours, readings taken every 15 seconds and Vmax calculated using SoftMax Pro v5.2 software.

FIGS. 26 and 27 show that the anti-Factor D antibody does not affect catalytic cleavage.

FIG. 28 is a hypothetical model depicting how an anti-Factor D antibody inhibits Factor B activation. The anti-Factor D antibody sterically inhibits docking of Factor D to factor B, preventing activation of factor B and formation of an active C3 convertase.

1. A crystal formed by a native sequence Factor D polypeptide or a functional fragment or conservative amino acid substitution variant thereof.

2. The crystal of claim 1 wherein the native sequence Factor D polypeptide is human or cynomolgous Factor D.

3. The crystal of claim 2 wherein the native sequence Factor D polypeptide is human Factor D of SEQ ID NO: 1.

4. The crystal of claim 3 characterized by unit cell parameters approximately equal to the following: cell dimensions a=132.048, b=132.048; c=180.288, space group P4₃2₁2, crystal constant: 2.4 Å, and R/Rfree=21.2%/27.2.

5. The crystal of claim 2 wherein the native sequence Factor D polypeptide is cynomolgous Factor D of SEQ ID NO: 2.

6. The crystal of claim 5 characterized by unit cell parameters approximately equal to the following: a=182.205; b=80.673; c=142.575; space group C2, crystal constant: 2.1 Å; and R/Rfree=21.1%/26.9.

7. A composition comprising a crystal of claim 1, claim 4 or claim 6.

8. A crystallizable composition comprising a Factor D polypeptide complexed with an anti-Factor D antibody or an antigen binding fragment of said antibody.

9. The crystallizable composition of claim 8, wherein said anti-Factor D antibody is a monoclonal antibody.

10. The crystallizable composition of claim 9 wherein said fragment is a Fab fragment.

11. The crystallizable composition of claim 9, wherein said Factor D polypeptide is human Factor D of SEQ ID NO: 1.

12. The crystallizable composition of claim 11 having the structure coordinates set forth in Appendix 1A.

13. The crystallizable composition of claim 9, wherein said Factor D polypeptide is cynomolgous Factor D of SEQ ID NO: 2.

14. The crystallizable composition of claim 13 having the structure coordinates set forth in Appendix 1B.

15. The crystallizable composition of claim 8 wherein said Factor D polypeptide comprises a catalytic triad.

16. A crystal comprising a Factor D polypeptide complexed with an anti-Factor D antibody or an antigen binding fragment thereof.

17. The crystal of claim 16 wherein said antibody is a monoclonal antibody.

18. The crystal of claim 17 wherein said fragment is a Fab fragment.

19. The crystal of claim 17 wherein the Factor D polypeptide is human Factor D of SEQ ID NO: 1.

20. The crystal of claim 19 having the structure coordinates of Appendix 1A.

21. The crystal of claim 17 wherein the Factor D polypeptide is cynomolgous Factor D of SEQ ID NO: 2.

22. The crystal of claim 21 having the structure coordinates of Appendix 1B.

23. The crystal of claim 16 wherein said Factor D polypeptide comprises a catalytic triad.

24. The crystal of claim 19 wherein in the human Factor D polypeptide of SEQ ID NO: 1, or antigen binding fragment thereof, one or more of amino acid residues D131, V132, P134, D165, R166, A167, T168, N170, R171, R172, T173, D176, G177, I179, E181, R222, and K223 participate in complexing with said anti-Factor D antibody.

25. The crystal of claim 24 wherein in the human Factor D polypeptide of SEQ ID NO: 1, or antigen binding fragment thereof, all of amino acid residues D131, V132, P134, D165, R166, A167, T168, N170, R171, R172, T173, D176, G177, I179, E181, R222, and K223 participate in complexing with said anti-Factor D antibody.

26. The crystal of claim 19 wherein in the human Factor D polypeptide of SEQ ID NO: 1, or antigen binding fragment thereof, amino acid residue R172 forms hydrogen bonds with the heavy and light chains of said anti-Factor D antibody of antigen binding fragment thereof.

27. A computer for producing a three-dimensional representation of: a molecular complex comprising a binding site defined by structure coordinates of amino acid residues D131, V132, P134, D165, R166, A167, T168, N170, R171, R172, T173, D176, G177, I179, E181, R222, and K223 of human Factor D of SEQ ID NO: 1, wherein said computer comprises: (i) a machine-readable data storage medium comprising a data storage material encoded with machine-readable data, wherein said data comprises the structure coordinates of amino acid residues D131, V132, P134, D165, R166, A167, T168, N170, R171, R172, T173, D176, G177, I179, E181, R222, and K223 of human Factor D of SEQ ID NO: 1, and (ii) instructions for processing said machine-readable data into said three-dimensional representation.

28. The computer of claim 27, further comprising a display for displaying said structure coordinates.

29. A method for evaluating the potential of a chemical entity to associate with a molecular complex comprising a binding site defined by structure coordinates of amino acid residues D131, V132, P134, D165, R166, A167, T168, N170, R171, R172, T173, D176, G177, I179, E181, R222, and K223 of human Factor D of SEQ ID NO: 1, comprising the steps of (i) employing computational means to perform a fitting operation between the chemical entity and said binding site of the molecular complex; and (ii) analyzing the results of said fitting operation to quantify the association between the chemical entity and said binding site.

30. The method of claim 29 wherein said chemical entity is an antibody or an antigen binding fragment thereof, or a peptide or small molecule mimetic of said antibody or antibody fragment.

31. The method of claim 30 wherein said antibody, or antigen binding fragment thereof, forms hydrogen bonds with one or more of said residues.

32. The method of claim 31 wherein said antibody, or antigen binding fragment thereof, forms hydrogen bonds with amino acid residue R172 of human Factor D of SEQ ID NO: 1.

33. A chemical entity identifiable by any of the methods of claims 29 to 32.

34. A computer for determining at least a portion of the structure coordinates corresponding to an X-ray diffraction pattern of a molecular complex, wherein said computer comprises: a) a machine-readable data storage medium comprising a data storage material encoded with machine-readable data, wherein said data comprises at least a portion of the structure coordinates according FIGS. 6 and 7 or Appendix 1a or 1b; b) a machine-readable data storage medium comprising a data storage material encoded with machine-readable data, wherein said data comprises an X-ray diffraction pattern of said molecular complex; c), a working memory for storing instructions for processing said machine-readable data of a) and b); d) a central processing unit coupled to said working memory and to said machine-readable data of a) and b) for performing a Fourier transform of the machine readable data of (a) and for processing said machine readable data of (b) into structure coordinates; and e) a display coupled to said central processing unit for displaying said structure coordinates of said molecular complex.


SEQUENCE LISTING (50 sequences)

SEQ ID NO: 1 - protein, 253 aa, Homo sapiens
MHSWERLAVLVLLGAAACAAPPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 2 - protein, 253 aa, Macaca fascicularis
MHSWERLAVLVLLGVAACAAQPRGRILGGREAEAHARPYMASVQVNGEHLCGGVLVAEQWVLSAAHCLEDAADGKVQVLLGAHSLSQPEPSKRLYDVLRAVPHPDSRPDTIDHDLLLLQLSEKATLGPAVRPLPWQRVDRDVEPGTLCDVAGWGIVSHAGRRPDRLQHVLLPVLDRATCNRRTHHDGAITQRMMCAESNRRDSCKGDSGGPLVCGGVLEGVVTSGSRVCGNRKKPGIYTRVASYAAWIDSVLA

SEQ ID NO: 3 - DNA, 1173 bp, Homo sapiens
gtgtctcagccacagcggcttcaccatgcacagctgggagcgcctggcagttctggtcctcctaggagcggccgcctgcgcggcgccgccccgtggtcggatcctgggcggcagagaggccgaggcgcacgcgcggccctacatggcgtcggtgcagctgaacggcgcgcacctgtgcggcggcgtcctggtggcggagcagtgggtgctgagcgcggcgcactgcctggaggacgcggccgacgggaaggtgcaggttctcctgggcgcgcactccctgtcgcagccggagccctccaagcgcctgtacgacgtgctccgcgcagtgccccacccggacagccagcccgacaccatcgaccacgacctcctgctgctacagctgtcggagaaggccacactgggccctgctgtgcgccccctgccctggcagcgcgtggaccgcgacgtggcaccgggaactctctgcgacgtggccggctggggcatagtcaaccacgcgggccgccgcccggacagcctgcagcacgtgctcttgccagtgctggaccgcgccacctgcaaccggcgcacgcaccacgacggcgccatcaccgagcgcttgatgtgcgcggagagcaatcgccgggacagctgcaagggtgactccgggggcccgctggtgtgcgggggcgtgctcgagggcgtggtcacctcgggctcgcgcgtttgcggcaaccgcaagaagcccgggatctacacccgcgtggcgagctatgcggcctggatcgacagcgtcctggcctagggtgccggggcctgaaggtcagggtcacccaagcaacaaagtcccgagcaatgaagtcatccactcctgcatctggttggtctttattgagcacctactatatgcagaaggggaggccgaggtgggaggatcattggatctcaggagttcgagatcagcatgggccacgtagcgcgactccatctctacaaataaataaaaaattagctgggcaattggcgggcatggaggtgggtgcttgtagttccagctactcaggaggctgaggtgggaggatgacttgaacgcaggaggctgaggctgcagtgagttgtgattgcaccactgccctccagcctgggcaacagagtgaaaccttgtctctctctacaaaaaaaaaaaaaaaa

SEQ ID NO: 4 - DNA, 800 bp, Macaca fascicularis
atgcacagctgggagcgcctggcagttctggtcctcctgggagtggccgcctgcgcggcgcagccccgcggtcggatcctgggcggcagagaggccgaggcccacgcgcggccctacatggcgtcggtgcaggtgaacggcgagcacctgtgcggcggcgtcctggtggccgagcagtgggtgctgagcgcggcgcactgcctggaggacgcggccgacgggaaggtgcaggttctcctgggcgcgcactccctgtcgcagccggagccctccaagcgcctgtacgacgtgctccgcgcagtgccgcacccggacagccggcccgacaccatcgaccacgacctcctcctgctgcagctgtccgagaaggccacgctgggccctgctgtgcgccccctgccctggcagcgcgtggatcgcgacgtggagccgggcactctctgcgacgtggccggctggggcatagtcagccacgcgggccgccgcccggaccgcctgcagcacgtgctcttgccagtgctggaccgcgccacctgcaaccggcgcacgcaccacgacggcgccatcacccagcgtatgatgtgcgcggagagcaaccgccgggacagctgcaaaggcgactccgggggcccgctggtgtgcgggggcgtgctcgagggcgtggtcacctcgggctcgcgagtttgcggcaaccgcaagaagcccgggatctacacgcgcgtggcgagctatgcggcctggatcgacagcgtcctggcctagtctagagtcgacctgcagaagcttggccgccatggccc

SEQ ID NO: 5 - protein, 107 aa, Artificial Sequence
  source: /note=“Description of Artificial Sequence Synthetic polypeptide”
DIQVTQSPSSLSASVRDRVTITCITSTDIDDDMNWYQQKPGKVPKLLISGGNTLRPGVPSRFSGSGSGTDFTLTISSLQPEDVATYYCLQSDNLPYTFGQGTKLEIK

SEQ ID NO: 6 - protein, 107 aa, Artificial Sequence
  source: /note=“Description of Artificial Sequence Synthetic polypeptide”
DIQVTQSPSSLSASVGDRVTITCITSTDIDDDMNWYQQKPGKVPKLLISGGNTLRPGVPSRFSGSGSGTDFTLTISSLQPEDVATYYCLQSDSLPYTFGQGTKLEIK

SEQ ID NO: 7 - protein, 107 aa, Artificial Sequence
  source: /note=“Description of Artificial Sequence Synthetic polypeptide”
DIQVTQSPSSLSASVGDRVTITCITSTDIDDDMNWYQQKPGKVPKLLISHGNTLRPGVPSRFSGSGSGTDFTLTISSLQPEDVATYYCLQSDSLPYTFGQGTKLEIK

SEQ ID NO: 8 - protein, 107 aa, Artificial Sequence
  source: /note=“Description of Artificial Sequence Synthetic polypeptide”
DIQVTQSPSSLSASVGDRVTITCITSTDIDDDMNWYQQKPGKVPKLLISDGNTIRPGVPSRFSGSGSGTDFTLTISSLQPEDVATYYCLQSDSIPYTFGQGTKLEIK

SEQ ID NO: 9 - DNA, 714 bp, Artificial Sequence
  source: /note=“Description of Artificial Sequence Synthetic polynucleotide”
atgaagaagaatattgcgttcctacttgcctctatgtttgtcttttctatagctacaaacgcgtatgctgatatccaggtgacccagtctccatcctccctgtctgcatctgtaggagaccgcgtcaccatcacttgcattaccagcactgatattgatgatgatatgaactggtatcagcagaaaccagggaaagttcctaagctcctgatctctggaggcaatactcttcgtcctggggtcccatctcggttcagtggcagtggatctgggacagatttcactctcaccatcagcagcctgcagcctgaagatgttgcaacttattactgtttgcaaagtgattctttgccgtacacgtttggccagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 10 - protein, 107 aa, Artificial Sequence [sequence omitted]

SEQ ID NO: 11 - protein, 23 aa, Artificial Sequence
  source: /note=“Description of Artificial Sequence Synthetic peptide”
DIQVTQSPSSLSASVGDRVTITC

SEQ ID NO: 12 - protein, 15 aa, Artificial Sequence
  source: /note=“Description of Artificial Sequence Synthetic peptide”
WYQQKPGKVPKLLIS

SEQ ID NO: 13 - protein, 32 aa, Artificial Sequence
  source: /note=“Description of Artificial Sequence Synthetic polypeptide”
GVPSRFSGSGSGTDFTLTISSLQPEDVATYYC

SEQ ID NO: 14 - protein, 10 aa, Artificial Sequence
  source: /note=“Description of Artificial Sequence Synthetic peptide”
FGQGTKVEIK

SEQ ID NO: 15 - protein, 11 aa, Artificial Sequence
  source: /note=“Description of Artificial Sequence Synthetic peptide”
ITSTDIDDDMN

SEQ ID NO: 16 - protein, 7 aa, Artificial Sequence
  source: /note=“Description of Artificial Sequence Synthetic peptide”
GGNTLRP

SEQ ID NO: 17 - protein, 9 aa, Artificial Sequence
  source: /note=“Description of Artificial Sequence Synthetic peptide”
LQSDSLPYT

SEQ ID NO: 18 - DNA, 741 bp, Artificial Sequence
  source: /note=“Description of Artificial Sequence Synthetic polynucleotide”
atgaaaaagaatatcgcatttcttcttgcatctatgttcgttttttctattgctacaaacgcgtacgctcaggtccagctggtgcaatctgggcctgagttgaagaagcctggggcctcagtgaaggtttcctgcaaggcttctggatacaccttcactaactatggaatgaactgggtgcgccaagcccctggacaagggcttgagtggatgggatggattaacacctacactggagagacaacatatgctgatgacttcaagggacggtttgtcttctccttggacacctctgtcagcacggcatatctgcagatcagcagcctcaaggctgaggacactgccgtgtattactgtgagcgcgaggggggggttaataactggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacataa

SEQ ID NO: 19 - protein, 223 aa, Artificial Sequence
  source: /note=“Description of Artificial Sequence Synthetic polypeptide”
QVQLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYPPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 25 aa, Artificial Sequence
  source: /note=“Description of Artificial Sequence Synthetic peptide”
QVQLVQSGPELKKPGASVKVSCKAS

SEQ ID NO: 21 - protein, 14 aa, Artificial Sequence
  source: /note=“Description of Artificial Sequence Synthetic peptide”
WVRQAPGQGLEWMG

SEQ ID NO: 22 - protein, 32 aa, Artificial Sequence
  source: /note=“Description of Artificial Sequence Synthetic polypeptide”
RFVFSLDTSVSTAYLQISSLKAEDTAVYYCER

SEQ ID NO: 23 - protein, 11 aa, Artificial Sequence
  source: /note=“Description of Artificial Sequence Synthetic peptide”
WGQGTLVTVSS

SEQ ID NO: 24 - protein, 10 aa, Artificial Sequence
  source: /note=“Description of Artificial Sequence Synthetic peptide”
GYTFTNYGMN

SEQ ID NO: 25 - protein, 17 aa, Artificial Sequence
  source: /note=“Description of Artificial Sequence Synthetic peptide”
WINTYTGETTYADDFKG

SEQ ID NO: 26 - protein, 6 aa, Artificial Sequence
  source: /note=“Description of Artificial Sequence Synthetic peptide”
EGGVNN

SEQ ID NO: 27 - protein, 108 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 28 - DNA, 714 bp, Artificial Sequence; identical to SEQ ID NO: 9
  source: /note=“Description of Artificial Sequence Synthetic polynucleotide”

SEQ ID NO: 29 - protein, 214 aa, Artificial Sequence
  source: /note=“Description of Artificial Sequence Synthetic polypeptide”
DIQVTQSPSSLSASVGDRVTITCITSTDIDDDMNWYQQKPGKVPKLLISGGNTLRPGVPSRFSGSGSGTDFTLTISSLQPEDVATYYCLQSDSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 741 bp, Artificial Sequence
  source: /note=“Description of Artificial Sequence Synthetic polynucleotide”
atgaaaaagaatatcgcatttcttcttgcatctatgttcgttttttctattgctacaaacgcgtacgctgaagtccagctggtgcaatctgggcctgagttgaagaagcctggggcctcagtgaaggtttcctgcaaggcttctggatacaccttcactaactatggaatgaactgggtgcgccaagcccctggacaagggcttgagtggatgggatggattaacacctacactggagagacaacatatgctgatgacttcaagggacggtttgtcttctccttggacacctctgtcagcacggcatatctgcagatcagcagcctcaaggctgaggacactgccgtgtattactgtgagcgcgaggggggggttaataactggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacataa

SEQ ID NO: 31 - protein, 223 aa, Artificial Sequence
  source: /note=“Description of Artificial Sequence Synthetic polypeptide”
EVQLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 32 - protein, 25 aa, Artificial Sequence
  source: /note=“Description of Artificial Sequence Synthetic peptide”
EVQLVQSGPELKKPGASVKVSCKAS

SEQ ID NO: 33 - protein, 10 aa, Artificial Sequence
  source: /note=“Description of Artificial Sequence Synthetic peptide”
FGQGTKVEIK

SEQ ID NO: 34 - protein, 11 aa, Artificial Sequence
  source: /note=“Description of Artificial Sequence Synthetic peptide”
ITSTDIDDDMN

SEQ ID NO: 35 - protein, 7 aa, Artificial Sequence
  source: /note=“Description of Artificial Sequence Synthetic peptide”
GGNTLRP

SEQ ID NO: 36 - protein, 9 aa, Artificial Sequence
  source: /note=“Description of Artificial Sequence Synthetic peptide”
LQSDSLPYT

SEQ ID NO: 37 - protein, 107 aa, Artificial Sequence [sequence omitted]

SEQ ID NO: 38 - protein, 32 aa, Artificial Sequence; identical to SEQ ID NO: 13
  source: /note=“Description of Artificial Sequence Synthetic polypeptide”

SEQ ID NO: 39 - protein, 108 aa, Artificial Sequence; identical to SEQ ID NO: 27
  source: /note=“Description of Artificial Sequence Synthetic polypeptide”

SEQ ID NO: 40 - protein, 107 aa, Artificial Sequence
  source: /note=“Description of Artificial Sequence Synthetic polypeptide”
DIQVTQSPSSLSASVGDRVTITCITSTDIDDDLNWYQQKPGKVPKLLISGGNTLRPGVPSRFSGSGSGTDFTLTISSLQPEDVATYYCLQSDSLPYTFGQGTKLEIK

SEQ ID NO: 41 - protein, 115 aa, Artificial Sequence
  source: /note=“Description of Artificial Sequence Synthetic polypeptide”
QVQLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSS

SEQ ID NO: 42 - protein, 4 aa, Homo sapiens
DRAT

SEQ ID NO: 43 - protein, 4 aa, Homo sapiens
NRRT

SEQ ID NO: 44 - protein, 115 aa, Artificial Sequence
  source: /note=“Description of Artificial Sequence Synthetic polypeptide”
QVQLVQSGPELKKPGASVKVSCKASGYTFTNYGMNWVKQAPGQGLEWMGWINTYTGETTYADDFKGRFVFSLDTSVSTAYLQISSLKAEDTATYYCEREGGVDNWGQGTLVTVSS

SEQ ID NO: 45 - protein, 115 aa, Artificial Sequence; identical to SEQ ID NO: 41
  source: /note=“Description of Artificial Sequence Synthetic polypeptide”

SEQ ID NO: 46 - protein, 115 aa, Artificial Sequence
  source: /note=“Description of Artificial Sequence Synthetic polypeptide”
QVQLVQSGPELKKPGASVKVSCKASGYTFTNYGLNWVRQAPGCGLEWMGWINTYTGETTYADDFKGRFVFSLDTSVSTAYLQISSLKAEDTAVYYCEREGGVNNWGQGTLVTVSS

SEQ ID NO: 47 - protein, 115 aa, Artificial Sequence
  source: /note=“Description of Artificial Sequence Synthetic polypeptide”
QVQLVQSGPELKKPGASVKVSCKASGYTFTSYGMNWVRQAPGQGLEWMGWINTYTGETTYADDFKGRFVFSLDTSVSTAYLQISSLKAEDTAVYYCEREGGVNNWGQGTLVTVSS

SEQ ID NO: 48 - protein, 214 aa, Artificial Sequence; identical to SEQ ID NO: 29
  source: /note=“Description of Artificial Sequence Synthetic polypeptide”

SEQ ID NO: 49 - protein, 23 aa, Artificial Sequence
  source: /note=“Description of Artificial Sequence Synthetic peptide”
DIQVTQSPSSLSASVGDRVTITC

SEQ ID NO: 50 - protein, 15 aa, Artificial Sequence
  source: /note=“Description of Artificial Sequence Synthetic peptide”
WYQQKPGKVPKLLIS